(19) United States
(12) Patent Application Publication
(10) Pub. No.: US 2003/0219784 A1
(43) Pub. Date: Nov. 27, 2003

(54) Systems and methods for analysis of agricultural products

(75) Inventors: Hon S. Ip, Madison, WI (US); Witold A. Ziarno, Ambler, PA (US); Glen Donald, Madison, WI (US)

Correspondence Address: Tanya A. Arenson, MEDLEN & CARROLL, LLP, Suite 350, 101 Howard Street, San Francisco, CA, 94105, US

(73) Assignee: Third Wave Technologies, Inc., Madison, WI (US)

(21) Appl. No.: 10/353,751

(22) Filed: Jan. 28, 2003

Related U.S. Application Data
(60) Provisional application No. 60/352,917, filed on Jan. 29, 2002.

(51) Int. Cl. C12Q 1/68; G06F 19/00; G01N 33/48; G01N 33/50

(52) U.S. Cl. 435/6; 702/20

(57) ABSTRACT

The present invention relates to systems and methods for the nucleic acid based analysis of agricultural products. In particular, the present invention relates to the determination of wheat grades using nucleic acid analysis. The present invention further provides a lateral flow strip apparatus for use in nucleic acid detection assays. The present invention thus provides improved methods of grading commercially important grains (e.g., wheat).

This application claims priority to U.S. provisional patent application serial No. 60/352,917 filed Jan. 29, 2002 and herein incorporated by reference in its entirety.

### FIELD OF THE INVENTION

The present invention relates to systems and methods for the nucleic acid based analysis of agricultural products. In particular, the present invention relates to the determination of wheat grades using nucleic acid analysis. The present invention further provides a lateral flow strip apparatus for use in nucleic acid detection assays.

### BACKGROUND

The market for grains, such as wheat, is a global market, with widespread distribution networks. Canada is one of the world's largest exporters of wheat. Wheat distribution in Canada is largely governed by the Canadian Wheat Board (CWB), which serves as the marketing agency for Western Canadian wheat and barley growers. Its role is to market these grains for the best possible price. All proceeds from sales, less the marketing costs, are passed back to farmers. With annual revenues of CDN $4 to 6 billion, it is one of the country's biggest export firms and one of the world's largest grain marketing organizations.

The CWB is the sole exporter of western Canadian wheat and barley. Canada's Parliament gave wheat and barley producers this monopoly so they would have more power and security in the marketplace. Instead of competing against one another, Canada's 110,000 wheat and barley farmers sell as one and therefore can command a higher price for their product.

The CWB uses a price pooling strategy. Pooling means that all sales are deposited into one of four pool accounts: wheat, durum wheat (used primarily for pasta production), feed barley or designated barley. This ensures that all farmers benefit equally, regardless of when their grain is sold during the crop year. All farmers delivering the same grade of wheat or barley will receive the same return at the end of the crop year.

Farmers get an initial or partial payment upon delivery, which is guaranteed by the Government of Canada. If returns to the pool exceed the sum of these total payments, then farmers receive a final payment. Should returns fall short, something that rarely happens, the federal government makes up the difference. As well, the government guarantees the CWB's borrowings. This allows the CWB to finance its operations at significantly lower rates of interest than if it was a private sector company.

The prices of wheat on the global market are largely determined by a grading scale, with higher grades fetching significantly higher prices. Current grading practices rely on visual inspection of wheat samples are thus laborious. An accurate, inexpensive, and user friendly method of determining grades is needed to allow for maximum prices for sellers of wheat.

### SUMMARY OF THE INVENTION

The present invention relates to systems and methods for the nucleic acid based analysis of agricultural products. In particular, the present invention relates to the determination of wheat grades using nucleic acid analysis. The present invention further provides a lateral flow strip apparatus for use in nucleic acid detection assays.

Accordingly, in some embodiments, the present invention provides a method of determining the grade of a wheat sample, comprising providing a wheat sample; detection assay components suitable for the detection of three or more properties of the wheat sample; and performing a detection assay with the detection assay components and the wheat sample. In some embodiments, the detection assay components are suitable for the detection of 5, preferable 10 or more, and even more preferably, 15 or more properties of the wheat sample. In some embodiments, the method further comprises the step of determining the grade of the wheat sample based on the results of the detection assay. In some embodiments, the three or more properties are selected from the group consisting of presence of contaminating organisms, presence of contaminating wheat, presence of contaminating plants, presence of contaminating seeds, and presence of genetically modified organisms. In some embodiments, the contaminating wheat is a different variety of wheat than the wheat sample. In some embodiments, the contaminating organisms include, but are not limited to, micro organisms and macro organisms. In some embodiments, the micro organisms are selected from the group including, but not limited to, ergot, sclerotinia, fusarium, smut, mildew, streak mold, and smudge. In some embodiments, the macro organisms are selected from the group including, but not limited to, grasshopper, sawfly, midge, and army worm. In some embodiments, the contaminating plants are selected from the group including, but not limited to, grass, rye, barley, tritcale, oats, oat groats, and wild oat groats. In some embodiments, the contaminating seeds are selected from the group including, but not limited to, ragweed, tartary buckwheat, rye grass, and wild oats. In some embodiments, the detection assay is selected from the group including, but not limited to, a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, and a sandwich hybridization assay. In some embodiments, the assay is performed using a lateral flow strip. In some embodiments, the hybridization assay is an INVADER assay.

The present invention additionally provides a method of detecting contaminating wheat in a wheat sample, comprising providing a wheat sample suspected of containing contaminating wheat; detection assay components suitable for the detection of one or more types of contaminating wheat is the sample, wherein the contaminating wheat is a different variety of wheat than the wheat sample; and performing a detection assay with the detection assay components and the wheat sample. In some embodiments, the detection assay components are suitable for the detection of 3 or more, preferably 5 or more types of contaminating wheat in the sample. In some embodiments, the method further comprises the step of determining the number and identity of the contaminating wheat present in the wheat sample. In some embodiments, the method further comprises the step of determining the grade of the wheat sample based on the results of the detection assay. In some embodiments, the method further comprises the step of detecting three or more properties of the wheat sample, wherein the three or more properties include, but are not limited to, the presence of contaminating organisms, presence of contaminating plants, presence of contaminating seeds, and presence of genetically modified organisms. In some embodiments, the contaminating organisms include, but are not limited to, micro organisms and macro organisms. In some embodiments, the micro organisms are selected from the group including, but not limited to, ergot, sclerotinia, fusarium, smut, mildew, streak mold, and smudge. In some embodiments, the macro organisms are selected from the group including, but not limited to, grasshopper, sawfly, midge, and army worm. In some embodiments, the contaminating plants are selected from the group including, but not limited to, grass, rye, barley, tritcale, oats, oat groats, and wild oat groats. In some embodiments, the contaminating seeds are selected from the group including, but not limited to, ragweed, tartary buckwheat, rye grass, and wild oats. In some embodiments, the detection assay is selected from the group including, but not limited to, a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, and a sandwich hybridization assay. In some embodiments, the assay is performed using a lateral flow strip. In some embodiments, the hybridization assay is an INVADER assay.

The present invention further provides a system for the grading of wheat, comprising a detection assay component configured for the generation of nucleic acid information for three or more properties of a wheat sample; and an information distribution component configured for the distribution of the nucleic acid information. In some embodiments, the three or more properties are selected from the group including, but not limited to, presence of contaminating organisms, presence of contaminating wheat, presence of contaminating plants, presence of contaminating seeds, and presence of genetically modified organisms. In some embodiments, the contaminating wheat is a different variety of wheat than the wheat sample. In some embodiments, the contaminating organisms include, but are not limited to, micro organisms and macro organisms. In some embodiments, the micro organisms are selected from the group including, but not limited to, ergot, sclerotinia, fusarium, smut, mildew, streak mold, and smudge. In some embodiments, the macro organisms are selected from the group including, but not limited to, grasshopper, sawfly, midge, and army worm. In some embodiments, the contaminating plants are selected from the group including, but not limited to, grass, rye, barley, tritcale, oats, oat groats, and wild oat groats. In some embodiments, the contaminating seeds are selected from the group including, but not limited to, ragweed, tartary buckwheat, rye grass, and wild oats. In some embodiments, the detection assay component comprises reagent for performing a detection assay selected from the group including, but not limited to, a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, and a sandwich hybridization assay. In some embodiments, the detection assay component is a lateral flow strip. In some embodiments, the information distribution component comprises a computer system, the computer system comprising a computer processor and computer memory. In some embodiments, the computer processor and computer memory are in communication with the Internet. In some embodiments, the computer system is in possession of a user including, but not limited to, a farmer, a distributor, and a customer.

The present invention also provides a system for the detection of nucleic acid sequences comprising, a lateral flow strip apparatus comprising a reaction well, a nucleic acid capture well, a label capture well, and an addressable detection section; reagents for the detection of nucleic acid sequences, the reagent in communication with the lateral flow strip apparatus. In some embodiments, the reagents comprise reagents for performing an INVADER assay. In some embodiments, the reagent comprises hybridization probes specific for the nucleic acid sequences, wherein each of the hybridization probes is specific for a unique nucleic acid sequence, and wherein each of the hybridization probes comprises a unique label. In some embodiments, the reagents comprise antibodies specific for each of the unique labels. In some preferred embodiments, the nucleic acid sequences are wheat nucleic acid sequences. In some embodiments, the nucleic acid sequences are sequences found in wheat contaminants.

### DESCRIPTION OF THE FIGURES

The following figures form part of the present specification and are included to further demonstrate certain aspects and embodiments of the present invention. The invention may be better understood by reference to one or more of these figures in combination with the description of specific embodiments presented herein.

FIG. 1 shows an overview of the Canadian grain distribution system.

FIG. 2 shows the lateral flow strip used in some embodiments of the present invention.

FIG. 3 shows an exemplary assay incubation format for use with the lateral flow strip apparatus in some embodiments of the present invention.

### DEFINITIONS

To facilitate an understanding of the present invention, a number of terms and phrases are defined below:

As used herein, the terms “solid support” or “support” refer to any material that provides a solid or semi-solid structure with which another material can be attached. Such materials include smooth supports (e.g., metal, glass, plastic, silicon, and ceramic surfaces) as well as textured and porous materials. Such materials also include, but are not limited to, gels, rubbers, polymers, and other non-rigid materials. Solid supports need not be flat. Supports include any type of shape including spherical shapes (e.g., beads). Materials attached to solid support may be attached to any portion of the solid support (e.g., may be attached to an interior portion of a porous solid support material). Preferred embodiments of the present invention have biological molecules such as nucleic acid molecules and proteins attached to solid supports. A biological material is “attached” to a solid support when it is associated with the solid support through a non-random chemical or physical interaction. In some preferred embodiments, the attachment is through a covalent bond. However, attachments need not be covalent or permanent. In some embodiments, materials are attached to a solid support through a “spacer molecule” or “linker group.” Such spacer molecules are molecules that have a first portion that attaches to the biological material and a second portion that attaches to the solid support. Thus, when attached to the solid support, the spacer molecule separates the solid support and the biological materials, but is attached to both.

As used herein, the term “treating together,” when used in reference to experiments or assays, refers to conducting experiments concurrently or sequentially, wherein the results of the experiments are produced, collected, or analyzed together (i.e., during the same time period). For example, a plurality of different target sequences located in separate wells of a multiwell plate or in different portions of a microarray are treated together in a detection assay where detection reactions are carried out on the samples simultaneously or sequentially and where the data collected from the assays is analyzed together.

The terms “assay data” and “test result data” as used herein refer to data collected from performance of an assay (e.g., to detect or quantitate a gene or other nucleic acid). Test result data may be in any form, i.e., it may be raw assay data or analyzed assay data (e.g., previously analyzed by a different process). Collected data that has not been further processed or analyzed is referred to herein as “raw” assay data (e.g., a number corresponding to a measurement of signal, such as a fluorescence signal from a spot on a chip, flow strip or a reaction vessel, or a number corresponding to measurement of a peak, such as peak height or area, as from, for example, a mass spectrometer, HPLC or capillary separation device), while assay data that has been processed through a further step or analysis (e.g., normalized, compared, or otherwise processed by a calculation) is referred to as “analyzed assay data” or “output assay data”.

As used herein, the term “database” refers to collections of information (e.g., data) arranged for ease of retrieval, for example, stored in a computer memory. “Database information” refers to information to be sent to databases, stored in a database, processed in a database, or retrieved from a database. “Sequence database information” refers to database information pertaining to nucleic acid sequences. As used herein, the term “distinct sequence databases” refers to two or more databases that contain different information than one another.

As used herein, the terms “computer memory” and “computer memory device” refer to any storage media readable by a computer processor. Examples of computer memory include, but are not limited to, random access memory (RAM), read only memory (ROM), computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refers to a device that is able to read a program from a computer memory (e.g., ROM or other computer memory) and perform a set of steps according to the program.

As used herein the term “oligonucleotide specification information” refers to any information used during the production of an oligonucleotide. Examples of oligonucleotide specification information includes, but is not limited to, sequence information, end-user (e.g., customer) information, and concentration information (e.g., the final concentration desired by the end-user).

As used herein the term “purified sample,” as in a purified oligonucleotide sample, refers to a sample where the full-length oligonucleotide in a sample is the predominate species of oligonucleotide. For example, in some embodiments, at least 90%, preferably 95%, and more preferably 99% of oligonucleotides in a sample are full-length oligonucleotides.

As used herein, the term “linkage” refers to the proximity of two or more markers (e.g., genes) on a chromosome.

As used herein, the term “genotype” refers to the actual genetic make-up of an organism (e.g., in terms of the particular alleles carried at a genetic locus). Expression of the genotype gives rise to an organism's physical appearance and characteristics—the “phenotype.”

As used herein, the term “locus” refers to the position of a gene or any other characterized sequence on a chromosome.

The term “gene” refers to a nucleic acid (e.g., DNA) sequence that comprises coding sequences necessary for the production of a polypeptide, RNA (e.g., rRNA, tRNA, etc.), or precursor. The polypeptide, RNA, or precursor can be encoded by a full length coding sequence or by any portion of the coding sequence so long as the desired activity or functional properties (e.g., ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments included when a gene is transcribed into heterogeneous nuclear RNA (hnRNA); introns may contain regulatory elements such as enhancers. Introns are removed or “spliced out” from the nuclear or primary transcript; introns therefore are generally absent in the messenger RNA (mRNA) transcript. The mRNA functions during translation to specify the sequence or order of amino acids in a nascent polypeptide. Variations (e.g., mutations, SNPS, insertions, deletions) in transcribed portions of genes are reflected in, and can generally be detected in corresponding portions of the produced RNAs (e.g., hnRNAs, mRNAs, rRNAs, tRNAs).

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. In this case, the DNA sequence thus codes for the amino acid sequence.

DNA and RNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in either a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids′ bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42 C when a probe of about 500 nucleotides in length is employed.

The following terms are used to describe the sequence relationships between two or more polynucleotides: “reference sequence,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

As applied to polynucleotides, the term “substantial identity” denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a splice variant of the full-length sequences.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q replicase, MDV-1 RNA is the specific template for the replicase (D. L. Kacian et al., Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (M. Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” or “hybridization probe” refers to an oligonucleotide (i.e., a sequence of nucleotides), whether occurring naturally as in a purified restriction digest or produced synthetically, recombinantly or by PCR amplification, that is capable of hybridizing, at least in part, to another oligonucleotide of interest. A probe may be single-stranded or double-stranded. Probes are useful in the detection, identification and isolation of particular sequences. In some preferred embodiments, probes used in the present invention will be labeled with a “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target” refers to a nucleic acid sequence or structure to be detected or characterized.

As used herein, the term “polymerase chain reaction” (“PCR”) refers to the method of K. B. Mullis (See e.g., U.S. Pat. Nos. 4,683,195, 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acids encoding a polypeptide include, by way of example, such nucleic acid in cells ordinarily expressing the polypeptide where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (e.g., 10 nucleotides, 11, . . . , 20, . . . ).

As used herein, the term “purified” or “to purify” refers to the removal of contaminants from a sample. As used herein, the term “purified” refers to molecules (e.g., nucleic or amino acid sequences) that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “sample” as used herein is used in its broadest sense. A sample suspected of containing a protein or nucleic acid sequence may comprise a cell, a portion of a tissue, an extract containing one or more proteins and the like.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable.

The term “signal” as used herein refers to any detectable effect, such as would be caused or provided by a label or an assay reaction.

As used herein, the term “detector” refers to a system or component of a system, e.g., an instrument (e.g. a camera, fluorimeter, charge-coupled device, scintillation counter, etc) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection system known in the art, or combinations thereof

As used herein, the term “distribution system” refers to systems capable of transferring and/or delivering materials from one entity to another or one location to another. For example, a distribution system for transferring detection panels from a manufacturer or distributor to a user may comprise, but is not limited to, a packaging department, a mail room, and a mail delivery system. Alternately, the distribution system may comprise, but is not limited to, one or more delivery vehicles and associated delivery personnel, a display stand, and a distribution center. In some embodiments of the present invention interested parties (e.g., detection panel manufactures) utilize a distribution system to transfer detection panels to users at no cost, at a subsidized cost, or at a reduced cost.

The term “detection” as used herein refers to quantitatively or qualitatively identifying an analyte (e.g., DNA, RNA or a protein) within a sample. The term “detection assay” as used herein refers to a kit, test, or procedure performed for the purpose of detecting an analyte nucleic acid within a sample. Detection assays produce a detectable signal or effect when performed in the presence of the target analyte, and include but are not limited to assays incorporating the processes of hybridization, nucleic acid cleavage (e.g., exo- or endonuclease), nucleic acid amplification, nucleotide sequencing, primer extension, or nucleic acid ligation.

As used herein, the term “functional detection oligonucleotide” refers to an oligonucleotide that is used as a component of a detection assay, wherein the detection assay is capable of successfully detecting (i.e., producing a detectable signal) an intended target nucleic acid when the functional detection oligonucleotide provides the oligonucleotide component of the detection assay. This is in contrast to a non-functional detection oligonucleotides, which fail to produce a detectable signal in a detection assay for the particular target nucleic acid when the non-functional detection oligonucleotide is provided as the oligonucleotide component of the detection assay. Determining if an oligonucleotide is a functional oligonucleotide can be carried out experimentally by testing the oligonucleotide in the presence of the particular target nucleic acid using the detection assay.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “hypertext system” refers to a computer-based informational system in which documents (and possibly other types of data entities) are linked together via hyperlinks to form a user-navigable “web.”

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc). The term is also intended to encompass non-public networks such as private (e.g., corporate) Intranets.

As used herein, the terms “World Wide Web” or “web” refer generally to both (i) a distributed collection of interlinked, user-viewable hypertext documents (commonly referred to as Web documents or Web pages) that are accessible via the Internet, and (ii) the client and server software components which provide user access to such documents using standardized Internet protocols. Currently, the primary standard protocol for allowing applications to locate and acquire Web documents is HTTP, and the Web pages are encoded using HTML. However, the terms “Web” and “World Wide Web” are intended to encompass future markup languages and transport protocols that may be used in place of (or in addition to) HTML and HTTP.

As used herein, the term “web site” refers to a computer system that serves informational content over a network using the standard protocols of the World Wide Web. Typically, a Web site corresponds to a particular Internet domain name and includes the content associated with a particular organization. As used herein, the term is generally intended to encompass both (i) the hardware/software server components that serve the informational content over the network, and (ii) the “back end” hardware/software components, including any non-standard or specialized components, that interact with the server components to perform services for Web site users.

As used herein, the term “HTML” refers to HyperText Markup Language that is a standard coding convention and set of codes for attaching presentation and linking attributes to informational content within documents. HTML is based on SGML, the Standard Generalized Markup Language. During a document authoring stage, the HTML codes (referred to as “tags”) are embedded within the informational content of the document. When the Web document (or HTML document) is subsequently transferred from a Web server to a browser, the codes are interpreted by the browser and used to parse and display the document. Additionally, in specifying how the Web browser is to display the document, HTML tags can be used to create links to other Web documents (commonly referred to as “hyperlinks”).

As used herein, the term “XML” refers to Extensible Markup Language, an application profile that, like HTML, is based on SGML. XML differs from HTML in that: information providers can define new tag and attribute names at will; document structures can be nested to any level of complexity; any XML document can contain an optional description of its grammar for use by applications that need to perform structural validation. XML documents are made up of storage units called entities, which contain either parsed or unparsed data. Parsed data is made up of characters, some of which form character data, and some of which form markup. Markup encodes a description of the document's storage layout and logical structure. XML provides a mechanism to impose constraints on the storage layout and logical structure, to define constraints on the logical structure and to support the use of predefined storage units. A software module called an XML processor is used to read XML documents and provide access to their content and structure.

As used herein, the term “HTTP” refers to HyperText Transport Protocol that is the standard World Wide Web client-server protocol used for the exchange of information (such as HTML documents, and client requests for such documents) between a browser and a Web server. HTTP includes a number of different types of messages that can be sent from the client to the server to request different types of server actions. For example, a “GET” message, which has the format GET, causes the server to return the document or file located at the specified URL.

As used herein, the term “URL” refers to Uniform Resource Locator that is a unique address that fully specifies the location of a file or other resource on the Internet. The general format of a URL is protocol://machine address:port/path/filename. The port specification is optional, and if none is entered by the user, the browser defaults to the standard port for whatever service is specified as the protocol. For example, if HTTP is specified as the protocol, the browser will use the HTTP default port of 80.

As used herein, the term “communication network” refers to any network that allows information to be transmitted from one location to another. For example, a communication network for the transfer of information from one computer to another includes any public or private network that transfers information using electrical, optical, satellite transmission, and the like. Two or more devices that are part of a communication network such that they can directly or indirectly transmit information from one to the other are considered to be “in electronic communication” with one another. A computer network containing multiple computers may have a central computer (“central node”) that processes information to one or more sub-computers that carry out specific tasks (“sub-nodes”). Some networks comprises computers that are in “different geographic locations” from one another, meaning that the computers are located in different physical locations (i.e., aren't physically the same computer, e.g., are located in different countries, states, cities, rooms, etc.).

As used herein, the term “detection assay component” refers to a component of a system capable of performing a detection assay. Detection assay components include, but are not limited to, hybridization probes, buffers, and the like.

As used herein, the term “a detection assay configured for target detection” refers to a collection of assay components that are capable of producing a detectable signal when carried out using the target nucleic acid. For example, a detection assay that has empirically been demonstrated to detect a particular single nucleotide polymorphism is considered a detection assay configured for target detection.

As used herein, the phrase “unique detection assay” refers to a detection assay that has a different collection of detection assay components in relation to other detection assays located on the same detection panel. A unique assay doesn't necessarily detect a different target (e.g. SNP) than other assays on the same detection panel, but it does have a least one difference in the collection of components used to detect a given target (e.g. a unique detection assay may employ a probe sequences that is shorter or longer in length than other assays on the same detection panel).

As used herein, the term “candidate” refers to an assay or analyte, e.g., a nucleic acid, suspected of having a particular feature or property. A “candidate sequence” refers to a nucleic acid suspected of comprising a particular sequence, while a “candidate oligonucleotide” refers to an oligonucleotide suspected of having a property such as comprising a particular sequence, or having the capability to hybridize to a target nucleic acid or to perform in a detection assay. A “candidate detection assay” refers to a detection assay that is suspected of being a valid detection assay.

As used herein, the term “detection panel” refers to a substrate or device containing at least two unique candidate detection assays configured for target detection.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, allele frequencies, RNA expression levels, protein expression, phenotypes correlating to genotypes, etc.

As used herein, the term “synthesis” refers to the assembly of polymers from smaller units, such as monomers.

As used herein, the term “parallel” refers to systems or actions functioning in an essentially simultaneous, side-by-side, manner (e.g., parallel synthesis or parallel synthesis system).

As used herein, the term “distinct” in reference to signals refers to signals that can be differentiated one from another, e.g., by spectral properties such as fluorescence emission wavelength, color, absorbance, mass, size, fluorescence polarization properties, charge, etc., or by capability of interaction with another moiety, such as with a chemical reagent, an enzyme, an antibody, etc.

### DETAILED DESCRIPTION OF THE INVENTION

The present invention relates to systems and methods for the nucleic acid based analysis of agricultural products. In particular, the present invention relates to the determination of wheat grades using nucleic acid analysis. The present invention further provides a lateral flow strip apparatus for use in nucleic acid detection assays.

The following discussion provides a description of certain preferred illustrative embodiments of the present invention and is not intended to limit the scope of the present invention. For convenience, the discussion focuses on the application of the present invention to the analysis of wheat, but it should be understood that the methods and systems are intended for use in the development of tools for the analysis of any agricultural product (e.g., cereal grains).

I. Grain Grading and Distribution

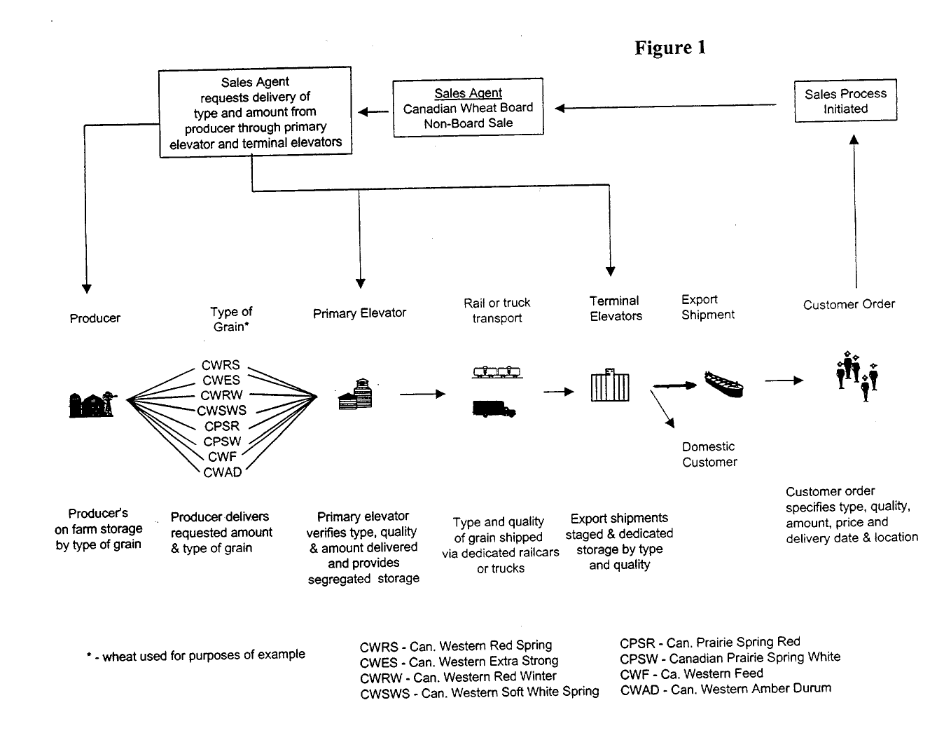

The Canadian wheat board governs the sale and distribution of wheat from Canada. An overview of the distribution and sale of Canadian wheat is shown in FIG. 1. Briefly, the producer (farmer) delivers the wheat to a primary elevator. The wheat is then transported to a terminal elevator near the export port. Finally, the wheat is transported (e.g., via an ocean) to customers. During the distribution process, the grain is graded and segregated via type and grade.

A. Grade Standards

The Canadian Grain Commision (CGC) sets grade standards for wheat. The Canada Grain Act provides for the appointment by the Commission of an Eastern Standards Committee and a Western Standards Committee. It specifies the numbers and qualifications of members. The Committees recommend specifications for grades of grain, select, recommend standard samples to the Commission, and perform any other related duties the Commission may assign. Their recommendations are forwarded to the Commission for consideration. Wide representation on the Standards Committees ensures that the views of all principals are considered before changes are made to the Canadian grading system. Grade definitions are changed only after thorough research and investigation have firmly established that meaningful changes would increase acceptability of Canadian grains in world markets.

Grades are assigned on the basis of visual quality characteristics of grain. Some visual characteristics can be measured objectively, while others are subjective (See e.g., the Official Grain Grading Guide (published Aug. 1, 2001 by the Canadian Grain Commission, herein incorporated by reference and also available in its entirety in U.S. provisional patent application serial No. 60/352,917 filed Jan. 29, 2002 and herein incorporated by reference) for detailed description of grain classes and criteria for each class). They are identified by their visual characteristics, called kernel visual distinguishability, KVD. New varieties must perform the same as or better than other varieties in the same class, and they must also look like other varieties within the same class. Similarly, grades within each class are visually distinguishable. Grain grades in Canada are built on qualities that customers want. Because customers' needs change, grades must be reviewed regularly. Under the Canada Grain Act, the Western and Eastern grain standards committees discuss and recommend specifications for grades of grain. Once the grain standards committees recommend a change, the CGC reviews the recommendation. If the CGC approves it, the recommendation is then published as a regulation in the Canada Gazette.

Standard samples reflect the conditions of the growing season. A standard sample is a sample of grain that represents the minimum visual quality for each grade of grain that will reach the marketplace in a given year. Slight variations in appearance from year to year reflect variations in environmental conditions from year to year. However, the standard sample maintains the processing quality for the class and grade.

To meet the demands of the marketplace, Canada began to market wheat on the basis of guaranteed protein levels in 1971. Protein segregations are available within higher grades of wheat-customers could get No. 1 CWRS, for example, in protein levels of 14.0%, 13.5%, 13.0%, etc. A quick test now available at major inspection points allows the protein to be measured and the wheat to be segregated.

B. Nucleic Acid-Based Analysis of Grade

In some embodiments, the present invention provides improved methods of grading wheat and other agricultural products. The nucleic acid-based grading methods of the present invention may be performed at any stage of the distribution process. For example, in some embodiments, grading is performed by the farmer on-site. In other embodiments, grading is performed at a primary or terminal elevator. In preferred embodiments, grading is performed prior to the wheat leaving Canada. However, the methods of the present invention are suitable for grading by the customer (e.g., local distributor or processor). The grading methods of the present invention are described in greater detail below.

The nucleic acid analysis methods of the present invention are applicable to the determination of many of the criteria used for grading. For example, the methods of the present invention are suitable for determining the variety of wheat. In preferred embodiments, the methods of the present invention are utilized to determine the presence of additional varieties of wheat in a sample in a single assay (e.g., by identifying a genetic marker or markers specific for a particular variety). In some preferred embodiments, the methods of the present invention provide for the determination of representative amounts of several varieties of wheat in a sample comprising a combination of varieties or grades of wheat.

In some embodiments, the methods of the present invention are used to determine damage to wheat by organisms. For example, in some embodiments, the amount of damage is determined by measuring the amount of contaminating organism in the sample. In some embodiments, the nucleic acid analysis methods of the present invention are used to determine the presence of microorganisms in wheat samples (e.g., including, but not limited to, ergot, sclerotinia, fusarium, smut, mildew, streak mold and smudge). In additional embodiments, the methods of the present invention are used to determine the amount of contaminating macro organisms in a sample (e.g., including, but not limited to, grasshopper, sawfly, midge, and army worm). In the case of macro organisms, visual comparisons can be used to determine the correlation between the amount of organism present at harvest and the expected damage.

In some embodiments, the presence of properties of the wheat kernels (e.g., frost damage, sprouted kernels, and immature kernels) is determined by measuring the expressing of nucleic acid (e.g., RNA or cDNA) corresponding to proteins associated with the particular property being measured (e.g. expression of genes that are activated in a cold-response or at a particular developmental stage).

In still further embodiments, the methods of the present invention are used to determine the amount of contaminating cereal grains including, but not limited to, rye barley, triticale, oats, oat groats, and wild oat groats. In yet other embodiments, the methods of the present invention are used to determine the amount of contaminating insepable seeds including, but not limited to, ragweed, tartary buckwheat, rye grass, and wild oats. In preferred embodiments, quantitative nucleic acid analysis is utilized to determine the amount of contaminating organisms. In yet other embodiments, the present invention provides methods for the detection of genetically modified organims (e.g., organims comprising exogenous nucleic acid sequences).

The below sections describe exemplary methods for nucleic acid analysis of wheat samples for the purposes of grading. The present invention is not limited to the analysis methods below. Indeed, the present invention includes all suitable methods of analysis.

II. Detection Assays

There are a wide variety of detection technologies available for determining the presence of nucleic acid sequences in wheat samples. Many of these techniques require the use of an oligonucleotide to hybridize to the target. Depending on the assay used, the oligonucleotide is then cleaved, elongated, ligated, disassociated, or otherwise altered, wherein its behavior in the assay is monitored as a means for characterizing the target nucleic acid. A number of these technologies are described in detail below.

While the systems and methods of the present invention are not limited to any particular detection assay, the following description illustrates the invention when used in conjunction with the INVADER assay (Third Wave Technologies, Madison Wis.; See e.g. U.S. Pat. Nos. 5,846,717; 6,090,543; 6,001,567; 5,985,557; 5,994,069, 6,214,545, 6,210,880, and 6,194,880; Lyamichev et al., Nat. Biotech., 17:292 (1999), Hall et al., PNAS, USA, 97:8272 (2000), Agarwal et al., Diagn. Mol. Pathol. 9:158 [2000], Cooksey et al., Antimicrob. Agents Chemother. 44:1296 [2000], Griffin and Smith, Trends Biotechnol., 18:77 [2000], Griffin and Smith, Analytical Chemistry 72:3298 [2000], Hessner et al., Clin. Chem. 46:1051 [2000], Ledford et al., J. Molec. Diagnostics 2,:97 [2000], Lyamichev et al., Biochemistry 39:9523 [2000], Mein et al., Genome Res., 10:330 [2000], Neri et al., Advances in Nucleic Acid and Protein Analysis 3826:117 [2000], Fors et al., Pharmacogenomics 1:219 [2000], Griffin et al., Proc. Natl. Acad. Sci. USA 96:6301 [1999], Kwiatkowski et al., Mol. Diagn. 4:353 [1999], and Ryan et al., Mol. Diagn. 4:135 [1999], Ma et al., J. Biol. Chem., 275:24693 [2000], Reynaldo et al., J. Mol. Biol., 297:511 [2000], and Kaiser et al., J. Biol. Chem., 274:21387 [1999]; and PCT publications WO97/27214, WO98/42873, and WO98/50403, each of which is herein incorporated by reference in their entirety for all purposes) to illustrate preferred features of the present invention) to detect a sequence of interest. The INVADER assay provides ease-of-use and sensitivity levels that, when used in conjunction with the systems and methods of the present invention, find use in the methods of the present invention. One skilled in the art will appreciate that specific and general features of this illustrative example are generally applicable to other detection assays.

A. INVADER Assay

The INVADER assay provides means for forming a nucleic acid cleavage structure that is dependent upon the presence of a target nucleic acid and cleaving the nucleic acid cleavage structure so as to release distinctive cleavage products. 5′ nuclease activity, for example, is used to cleave the target-dependent cleavage structure and the resulting cleavage products are indicative of the presence of specific target nucleic acid sequences in the sample. When two strands of nucleic acid, or oligonucleotides, both hybridize to a target nucleic acid strand such that they form an overlapping invasive cleavage structure, as described below, invasive cleavage can occur. Through the interaction of a cleavage agent (e.g., a 5′ nuclease) and the upstream oligonucleotide, the cleavage agent can be made to cleave the downstream oligonucleotide at an internal site in such a way that a distinctive fragment is produced.

The INVADER assay provides detections assays in which the target nucleic acid is reused or recycled during multiple rounds of hybridization with oligonucleotide probes and cleavage of the probes without the need to use temperature cycling (i.e., for periodic denaturation of target nucleic acid strands) or nucleic acid synthesis (i.e., for the polymerization-based displacement of target or probe nucleic acid strands). When a cleavage reaction is run under conditions in which the probes are continuously replaced on the target strand (e.g. through probe-probe displacement or through an equilibrium between probe/target association and disassociation, or through a combination comprising these mechanisms, (Reynaldo, et al., J. Mol. Biol. 97: 511-520 [2000]), multiple probes can hybridize to the same target, allowing multiple cleavages, and the generation of multiple cleavage products.

The INVADER assay, as well as other assays, may also employ degenerate oligonucleotides (e.g. degenerate INVADER and probe oligonucleotides). For example, standard INVADER oligonucleotides and probes may be randomly changed at one more positions such that a set of degenerate INVADER and/or probe oligonucleotides are produced. Degenerate sets of INVADER and probe oligonucleotides are particularly useful for use in conjunction with target sequences that tend to be heavily mutated. Using such degenerate sets of INVADER and probe oligonucleotides allows the presence of target sequences at a particular location to be detected even if the surrounding sequence no longer represent the wild type or expected sequence.

The INVADER assay technology may be used to quantitate mRNA (e.g. without target amplification). Low variability (3-10% coefficient of variation) provides accurate quantitation of less than two-fold changes in mRNA levels. A biplex FRET-based detection format enables simultaneous quantitation of expression from two genes within the same sample. One of these genes can be an invariant housekeeping gene that is used as the internal standard. Normalizing the signals from the gene of interest with the internal standard provides accurate results and obviates the need for replicate samples. A simple and rapid cell lysate sample preparation method can be used with the mRNA INVADER Assay. The combined features of biplex detection and easy sample preparation make this assay readily adaptable for use in high-throughput applications.

In certain embodiments, the INVADER assay (and other detection assays such as TAQMAN) employ an E-TAG label (e.g. as part of the INVADER oligonucleotide, probe oligonucleotide, or the FRET oligonucleotide). E-TAG labeling is particularly useful in muliplex analysis. E-TAG labeling does not require surface immobilization of affinity agents. E-TAG type labeling is described in U.S. Pat. Nos. 5,858,188; 5,883,211; 5,935,401; 6,007,690; 6,043,036; 6,054,034; 6,056,860; 6,074,827; 6,093,296; 6,103,199; 6,103,537; 6,176,962; and 6,284,113, all of which are herein incorporated by reference.

1. Oligonucleotide Design for the INVADER Assay

The application of the INVADER assay is not limited to any particular type of nucleic acid or nucleic acid variations. In some embodiments, oligonucleotides for an INVADER assay are designed to detect a particular target sequence. In other embodiments, the oligonucleotides for an assay may be designed to determine the presence or absence of a particular nucleic acid in a sample, e.g., a nucleic acid suspected to be present as a consequence of, for example, transfection, transformation or infection of the source of the sample. In yet other embodiments, the oligonucleotides of an INVADER assay may be designed to provide quantitative information about a particular DNA or RNA sequence.

In some embodiments where an oligonucleotide is designed for use in the INVADER assay, the sequence(s) of interest are entered into the INVADERCREATOR program (Third Wave Technologies, Madison, Wis.). One skilled in the art will appreciate that applicability of aspects of this design system for use in other detection assays. As described above, sequences may be input for analysis from any number of sources, either directly into the computer hosting the INVADERCREATOR program, or via a remote computer linked through a communication network (e.g., a LAN, Intranet or Internet network). For detection of double-stranded nucleic acid, e.g., a gene, the program designs probes for both strands, e.g., the sense and antisense strands. Selection of a particular strand for detection is generally based upon factors that include the ease of synthesis, minimization of secondary structure formation, manufacturability and INVADERCREATOR penalty scores, which have been established by studying probe design performance in the INVADER assay. In some embodiments, the user chooses the strand for sequences to be designed for. In other embodiments, the software automatically selects the strand. By incorporating thermodynamic parameters for optimum probe cycling and signal generation (e.g., Allawi and SantaLucia, Biochemistry, 36:10581 [1997] for DNA duplexes, Sugimoto, et al., Biochemistry 34, 11211 [1995] for RNA/DNA hybrids, or Xia, et al., Biochemistry 37:14719 [1998], for RNA duplexes), oligonucleotide probes may be designed to operate at a pre-selected assay temperature (e.g., 63° C.). Based on these criteria, a final probe set (e.g., primary probes for 2 alleles and an INVADER oligonucleotide for a detection assay, or primary probe, a stacker oligonucleotide, an INVADER oligonucleotide and an ARRESTOR oligonucleotide for an RNA detection assay) is selected.

In some embodiments, the INVADERCREATOR system is a web-based program with secure site access and that can be linked to RNAstructure (Mathews et al., RNA 5:1458 [1999]), a software program that utilizes mfold (Zuker, Science, 244:48 [1989]). RNAstructure can test the proposed oligonucleotide designs generated by INVADERCREATOR for potential uni- and bimolecular complex formation. INVADERCREATOR is open database connectivity (ODBC)-compliant and uses the Oracle database for export/integration. The INVADERCREATOR system is configured with ORACLE to work well with UNIX systems, as most genome centers are UNIX-based.

Each INVADER reaction includes at least two target sequence-specific, unlabeled oligonucleotides for the primary reaction: an upstream INVADER oligonucleotide and a downstream Probe oligonucleotide. The INVADER oligonucleotide is generally designed to bind stably at the reaction temperature, while the probe is designed to freely associate and disassociate with the target strand, with cleavage occurring only when an uncut probe hybridizes adjacent to an overlapping INVADER oligonucleotide. In some embodiments, the probe includes a 5′ flap or “arm” that is not complementary to the target, and this flap is released from the probe when cleavage occurs. In some embodiments, the released flap participates as an INVADER oligonucleotide in a secondary reaction. In some embodiments, the INVADER reaction may comprise additional oligonucleotides, such as stacker or ARRESTOR oligonucleotides. In some embodiments, the designed oligonucleotides are submitted as a synthesis order, such that manufacture of each oligonucleotide is initiated at order submission, are tracked through the modules of synthesis and the manufactured set of oligonucleotides are collected into a finished assay product or kit. In other embodiments, the oligonucleotide designs are checked against an inventory of existing oligonucleotides to determine if any of the oligonucleotides of the assay have been previously synthesized (“pre-synthesized” oligonucleotides) and stored. In some embodiments, one or more pre-synthesized oligonucleotides are taken from inventory oligonucleotides and included with newly designed and synthesized oligonucleotides in the finished assay or kit. In other embodiments, new assays or kits are assembled entirely from pre-synthesized oligonucleotides taken from an inventory of oligonucleotides.

In some embodiments, of an INVADERCREATOR program, the program is configured to design oligonucleotides for an assay of a single particular type or purpose (e.g., for wheat analysis). In other embodiments, an INVADERCREATOR program is configured to allow a user to select, e.g., through a button, check box or menu, from a variety of assay types or purposes. The following discussion provides several examples of how a user interface for an INVADERCREATOR program may be configured.

In some embodiments, screens provide optional selection of any number of modifications (e.g., arms, dyes, detectable moieties) for detection or further manipulation. In some embodiments, an INVADERCREATOR module may be customized for a particular assay, or for the needs of a particular user or customer. For example, if a customer has a particular detection platform requiring that the cleavage products comprise moiety X, an INVADERCREATOR module can be configured such that all assays designed by or for customer X are automatically configured to comprise moiety X, in accordance with the customer's requirements. In some embodiments, a pre-designated design feature cannot be altered by an operator creating a new probe design using the customized INVADERCREATOR module. In other embodiments, a pre-designated design feature may be presented to an operator as a default condition of the design that may be overridden during probe design (e.g., by selecting an alternative configuration through one or more data entry screens).

In one embodiment of an INVADERCREATOR program, the user initiates oligonucleotide design by opening a work screen, e.g., by clicking on an icon on a desktop display of a computer (e.g., a Windows desktop). In some embodiments, the user enters information related to the assay, such as project code, company name, assay name, etc. In some embodiments, the user indicates what species the nucleic acid sequence is from. In some embodiments, the user selects the INVADERCREATOR program module to be used (e.g., SIC, RIC, TIC, etc.), e.g., by clicking a button on the screen. The user enters information related to the target sequence for which an assay is to be designed. In some embodiments, the user enters a target sequence. In other embodiments, the user enters a code or number that causes retrieval of a sequence from a database. In still other embodiments, additional information may be provided, such as the user's name, an identifying number associated with a target sequence, and/or an order number. In preferred embodiments, the user indicates (e.g. via a check box or drop down menu) that the target nucleic acid is DNA or RNA. In other preferred embodiments, the user indicates the species from which the nucleic acid is derived. In particularly preferred embodiments, the user indicates whether the design is for monoplex (i.e., one target sequence or allele per reaction) or multiplex (i.e., multiple target sequences or alleles per reaction) detection. When the requisite choices and entries are complete, the user starts the analysis process. In one embodiment, the user clicks a “Design It” button to continue.

In some embodiments, the software validates the field entries before proceeding. In some embodiments, the software verifies that any required fields are completed with the appropriate type of information. In other embodiments, the software verifies that the input sequence meets selected requirements (e.g., minimum or maximum length, DNA or RNA content). If entries in any field are not found to be valid, an error message or dialog box may appear. In preferred embodiments, the error message indicates which field is incomplete and/or incorrect. Once a sequence entry is verified, the software proceeds with the assay design.

In some embodiments, the information supplied in the order entry fields specifies what type of design will be created. In preferred embodiments, the target sequence and multiplex check box specify which type of design to create. Design options include but are not limited to SNP assay, Multiplexed SNP assay (e.g., wherein probe sets for different alleles are to be combined in a single reaction), Multiple SNP assay (e.g., wherein an input sequence has multiple sites of variation for which probe sets are to be designed), and Multiple Probe Arm assays.

In some embodiments, the INVADERCREATOR software is started via a Web Order Entry (WebOE) process (i.e., through an Intra/Internet browser interface) and these parameters are transferred from the WebOE via applet <param> tags, rather than entered through menus or check boxes.

In the case of Multiple SNP Designs, the user chooses two or more designs to work with. In some embodiments, this selection opens a new screen view. In some embodiments, the software creates designs for each locus specified in the target sequence, scoring each, and presents them to the user in this screen view. The user can then choose any two designs to work with. In some embodiments, the user chooses a first and second design (e.g., via a menu or buttons) and clicks a “Design It” button to continue.

To select a probe sequence that will perform optimally at a pre-selected reaction temperature, the melting temperature (Tₘ) of the SNP to be detected is calculated using the nearest-neighbor model and published parameters for DNA duplex formation (Allawi and SantaLucia, Biochemistry, 36:10581 [1997], SantaLucia, Proc Natl Acad Sci USA., 95(4):1460 [1998]). In embodiments wherein the target strand is RNA, parameters appropriate for RNA/DNA heteroduplex formation may be used. Because the assay's salt concentrations are often different than the solution conditions in which the nearest-neighbor parameters were obtained (1M NaCl and no divalent metals), an adjustment should be made to the value provided for the salt concentration within the melting temperature calculations. This adjustment is termed a ‘salt correction’ SantaLucia, Proc Natl Acad Sci USA., 95(4):1460 [1998]. Similarly, the presence and concentration of the enzyme influence optimal reaction temperature. One way of compensating for these additional factors is to further vary the salt value in the Tm calculations. As used herein, the term “salt correction” refers to a variation made in the value provided for a salt concentration for the purpose of reflecting the effect on a Tₘ calculation for a nucleic acid duplex of a both an alternative salt effect and a non-salt parameter or condition affecting said duplex. Variation of the values provided for the strand concentrations will also affect the outcome of these calculations. By using a value of 0.5 M NaCl (SantaLucia, Proc Natl Acad Sci USA, 95:1460 [1998]) and strand concentrations of about 1 μM of the probe and 1 fM target, the algorithm used for calculating probe-target melting temperature has been adapted for use in predicting optimal INVADER assay reaction temperatures. For one set of 30 probes, the average deviation between optimal assay temperatures calculated by this method and those experimentally determined is about 1.5° C.

The length of the target-complementary region of a probe (e.g., the probe to a given SNP) is defined by the temperature selected for running the reaction (e.g., 63° C.). Starting from the target base that is paired to the probe nucleotide 5′ of the intended cleavage site (e.g., the position of the variant nucleotide on the target DNA)), and adding on the 3′ end, an iterative procedure is used by which the length of the target-binding region of the probe is increased by one base pair at a time until a calculated optimal reaction temperature (Tₘ plus salt correction to compensate for enzyme effect) matching the desired reaction temperature is reached. For INVADER assays detecting DNA targets, the non-complementary arm of the probe is preferably selected to allow the secondary reaction to cycle at the same reaction temperature. The entire probe oligonucleotide is screened using programs such as mfold (Zuker, Science, 244: 48 [1989]) or Oligo 5.0 (Rychlik and Rhoads, Nucleic Acids Res, 17: 8543 [1989]) for the possible formation of dimer complexes or secondary structures that could interfere with the reaction. The same principles are also followed for INVADER oligonucleotide design. Briefly, starting from the position N on the target DNA, additional residues complementary to the target DNA starting from residue N-1 are then added in the 5′ direction until the stability of the INVADER oligonucleotide-target hybrid exceeds that of the probe (and therefore the planned assay reaction temperature), generally by 15-20° C. The 3′ end of the INVADER oligonucleotide is designed to have a nucleotide not complementary to either allele suspected of being contained in the sample to be tested. The mismatch does not adversely affect cleavage (Lyamichev et al., Nature Biotechnology, 17: 292 [1999]), and it can enhance probe cycling, presumably by minimizing coaxial stabilization effects between the two probes.

It is one aspect of the assay design that all of the probe sequences may be selected to allow the primary and secondary reactions to occur at the same optimal temperature, so that the reaction steps can run simultaneously. In an alternative embodiment, the probes may be designed to operate at different optimal temperatures, so that the reaction steps are not simultaneously at their temperature optima.

In some embodiments, the software provides the user an opportunity to change various aspects of the design including but not limited to: probe, target and INVADER oligonucleotide temperature optima and concentrations; blocking groups; probe arms; dyes, capping groups and other adducts; individual bases of the probes and targets (e.g., adding or deleting bases from the end of targets and/or probes, or changing internal bases in the INVADER and/or probe and/or target oligonucleotides). In some embodiments, changes are made by selection from a menu. In other embodiments, changes are entered into text or dialog boxes. In preferred embodiments, this option opens a new screen.

In some embodiments, the software provides a scoring system to indicate the quality (e.g., the likelihood of performance) of the assay designs. In one embodiment, the scoring system includes a starting score of points (e.g., 100 points) wherein the starting score is indicative of an ideal design, and wherein design features known or suspected to have an adverse affect on assay performance are assigned penalty values. Penalty values may vary depending on assay parameters other than the sequences, including but not limited to the type of assay for which the design is intended (e.g., DNA, RNA, monoplex, multiplex) and the temperature at which the assay reaction will be performed. The following example provides illustrative scoring criteria for use with some embodiments of the INVADER assay based on an intelligence defined by experimentation.

Examples of design features in assays for DNA detection that may incur score penalties (e.g., SIC and TIC module penalties) include but are not limited to the following [penalty values are indicated in brackets; if there are 2 numbers, the first number is for lower temperature assays (e.g., 62-64° C.), second is for higher temperature assays (e.g., 65-66° C.)]:

1. [20] 3′ four bases of the INVADER oligonucleotide resembles the probe arm, for example:

| | PENALTY AWARDED IF |
|---|---|
| ARM SEQUENCE | INVADER ENDS IN: |
| Arm 1: CGCGCCGAGG | 5′.......GAGGX |
| | or 5′.......GAGGXX |
| Arm 2: ATGACGTGGCAGAC | 5′.......AGACX |
| | or 5′.......AGACXX |
| Arm 3: ACGGACGCGGAG | 5′.......GGAGX |
| | or 5′.......GGAGXX |
| Arm 4: TCCGCGCGTCC | 5′.......GTCCX |
| | or 5′.......GTCCXX |

2. [100] 3′ five bases of the INVADER oligonucleotide resembles the probe arm. for example:

| | PENALTY AWARDED IF |
|---|---|
| ARM SEQUENCE | INVADER ENDS IN: |
| Arm 1: CGCGCCGAGG | 5′.......CGAGGX |
| | or 5′.......CGAGGXX |
| Arm 2: ATGACGTGGCAGAC | 5′.......CAGACX |
| | or 5′.......CAGACXX |
| Arm 3: ACGGACGCGGAG | 5′.......CGGAGX |
| | or 5′.......CGGAGXX |
| Arm 4: TCCGCGCGTCC | 5′.......CGTCCX |
| | or 5′.......CGTCCXX |

3. [70] probe has a 5-base stretch containing the polymorphism

4. [60] probe has a 5-base stretch adjacent to the polymorphism

5. [15] probe has a 4-base stretch of Gs containing the polymorphism

6. [50] probe has a 5-base stretch of Gs—penalty added anytime it is infringed

7. [40] INVADER oligonucleotide 6-base stretch is of Gs—additional penalty

8. [90] two or three base sequence repeats at least four times starting in the region +1 to +4 of the probe.

9. [100] degenerate base occurs in the probe four bases from either end.

10. [100] probe hybridizing region is short ≦12 bases regardless of assay temperature.

11. [40] probe hybridizing region is long (≧26 bases).

12. [5] hybridizing region length exceeding 26—per base additional penalty

13, [80] insertion/deletion design with poor discrimination in first 3 bases after probe arm

14. [100] calculated INVADER oligonucleotide Tm<7.5C of probe target Tm

15. [100] a probe has a calculated Tm 2C less than its target Tm

Tie Breaker Rules for SIC Module:

1. If calculated probes Tms differ by more than 2.0C, then pick other strand for design.

2. If target of one strand 8 bases longer than that of other strand, then pick shorter strand.

Examples of design features in assays for RNA detection (e.g., RIC module penalties) that may incur score penalties include but are not limited to the following:

1. [50+25 increment/additional G] probe has 4-G stretch in the INVADER oligonucleotide, probe, or stacker.

2. [70] probe has 5-base stretch containing position 1

3. [60] probe has 5-base stretch containing position 2

4. [90] two or three base sequence repeats at least four times starting at position +1 in the probe

5. [100] probe hybridizing region is short (8 bases with a stacker or ≦12 bases without a stacker)

6. [40+5 increment/base] probe hybridizing region is long (≧17 bases with a stacker or ≧20 bases without a stacker)

7. [100] penultimate 3′ base of the INVADER oligonucleotide matches the 3′ base of the probe arm

In some embodiments, penalties are assessed for location of variations at or near the cleavage site. In other embodiments, penalties are assessed based on cleavage site base preferences (e.g., some enzyme may cleave after more efficiently after particular bases, such as Gs, and penalties may be used when a different base is placed in that location). In still other embodiments, penalties are assessed based on ranking of stacking interactions between a probe 3′ base and a stacking oligonucleotide 5′ base (e.g., in some embodiments, AA stacks may perform better than TT stacks.

In particularly preferred embodiments, temperatures for each of the oligonucleotides in the designs are recomputed and scores are recomputed as changes are made. In some embodiments, score descriptions can be seen by clicking a “descriptions” button. In some embodiments, a BLAST search option is provided. In preferred embodiments, a BLAST search is done by clicking a “BLAST Design” button. In some embodiments, this action brings up a dialog box describing the BLAST process. In preferred embodiments, the BLAST search results are displayed as a highlighted design on a Designer Worksheet.

In some embodiments, a user accepts a design by clicking an “Accept” button. In other embodiments, the program approves a design without user intervention. In preferred embodiments, the program sends the approved design to a next process step (e.g., into production; into a file or database). In some embodiments, the program provides a screen view (e.g., an Output Page), allowing review of the final designs created and allowing notes to be attached to the design. In preferred embodiments, the user can return to the Designer Worksheet (e.g., by clicking a “Go Back” button) or can save the design (e.g., by clicking a “Save It” button) and continue (e.g., to submit the designed oligonucleotides for production).

In some embodiments, the program provides an option to create a screen view of a design optimized for printing (e.g., a text-only view) or other export (e.g., an Output view). In preferred embodiments, the Output view provides a description of the design particularly suitable for printing, or for exporting into another application (e.g., by copying and pasting into another application). In particularly preferred embodiments, the Output view opens in a separate window.

2. TAQMAN Probe and Primer Design

A number of different strategies can be used to design TaqMan (5′ Nuclease assay) Probes. The following are example of considerations that may be used when designing TAQMAN probes. One consideration is to design PCR primers such that the amplicon size is between 50-150 base pairs. Another consideration is to design PCR primers that have a Tm of around 60° C., with less than 2° C. difference in Tm between forward and reverse primers. Preferred primers have GC % around 40-60% and have three or less consecutive runs of any nucleotide. Preferably, the primers have total lengths of between 18-25 nucleotides in length. PCR Primers are designed to have minimal haripin and minimal dimer formation tendencies (See below). Following selection of the PCR primers, the TAQMAN probe is then chosen from within the amplicon region, and has a Tm of about 10° C. higher than the Tm of the PCR primers (typically, 70° C.). TAQMAN probes should have a 5° FAM and a 3′ TAMRA (or other labels), and not begin with G. TAQMAN probes may be chosen, for example, by using programs such as OligoWalk to scan through the amplicon sequence and a probe chosen based upon predicted most stable thermodynamic parameters. Moreover, candidate TAQMAN probes can be eliminated which forms more than three consecutive basepairs with the PCR primers.

3. Multiplex PCR Primer Design

The INVADER assay can be used for the detection of single nucleotide polymorphisms (SNPs) with as little as 100-10 ng of genomic DNA without the need for target pre-amplification. However, if sample is in short supply, or nucleic acid is difficult to extract, the amount of sample DNA becomes a limiting factor for large-scale analysis.

In some embodiments, it may be desired to detect related loci in a multiplex PCR reaction. In some such embodiments, the similarity between loci may prevent or complicate detection assay analysis of the sequence, as the detection assay technology may not be able to sufficiently discriminate between the closely related sequences. The present invention provides methods to overcome such problems, by generating a unique target sequence using a nucleic acid amplification technique (e.g., PCR), such that the unique target sequence is tested by the detection assay, rather the original sample (e.g., genomic DNA). This method is compatible with multiplexing, where considerations are made to ensure that amplified target sequence meets several criteria: 1) that the target sequence contains the polymorphism to be analyzed; 2) that the target sequence represents a unique target sequence (i.e., it is the only sequence in the reaction mixture that is detected by a detection assay designed to target the target sequence); and 3) that the target sequence does not contain other polymorphisms that are detected by any of the detection assays present in the multiplex reaction. Suitable detection assay components may be selected with methods similar to those described above for the INVADERCREATOR methods. For example, in some embodiments, the software performs a BLAST alignment of the target sequence used for the assay to find similar sequences in the genome that may generate the cross-reactivity signal. The design of PCR primers with software program should prevent amplification of any of the similar loci except the locus containing the target. To avoid pre-amplification of sequences other than the specific target sequence, the software performs a BLAST alignment of the sequence amplified with a pair of primers against all other detection assay sequences included in the pool. If cross-reactivity or potential cross-reactivity exists, the set of primers is redesigned or the co-amplified sequences are included in different pools.

The same type of design analysis may be used for detection assays directed at the detection of haplotypes. For example, primers are generated to amplify sets of target sequences that each uniquely contain the polymorphisms to be detected.

In some embodiments, multiplex detection assays are provided in a plurality of arrays. For example, in some embodiments, a first array comprises assays configured for detection directly from genomic DNA and a second array comprises assays configured for pre-amplification of target sequences from genomic DNA prior to detection assay analysis of the target sequence.

In some preferred embodiments, only limited pre-amplification of target sequences is carried out prior to detection by the detection assay. For example, in some embodiments, only a 10⁵-10⁶ fold or less increase in target copy number is obtained prior to detection. This is in contrast to typical PCR reactions where 10¹⁰-10¹² or more fold amplification is utilized in detection reactions. In certain embodiments, 100 genotypes from a single PCR amplification are possible with the methods and systems of the present invention using only 10 ng of genomic DNA.

In some embodiments, kits are provided for pre-amplification and detection of target sequences. In some embodiments, the kits comprise amplification primers. For multiplex reactions, the amplification primers may be provided in a single container. The amplification primers may also be packaged with detection assay components. In some embodiments, amplification primers and detection assay components (e.g., INVADER assay components) are provided in a single container (e.g., in a single well of a multiwell plate). In some embodiments, the reaction components are provided in dry form in a reaction chamber. In some such embodiments, the kits are configured to allow reactions to occur where the only thing that is added to the reaction chamber is a solution containing genomic DNA.

The present invention provides methods and selection criteria that allow primer sets for multiplex PCR to be generated (e.g. that can be coupled with a detection assay, such as the INVADER assay). In some embodiments, software applications of the present invention automated multiplex PCR primer selection, thus allowing highly multiplexed PCR with the primers designed thereby.

The multiplex primer design systems may be employed to design PCR primer sets useful with a particular type of assay, such as the INVADER assay. In some embodiments, the selection of primers to make a primer set capable of multiplex PCR is performed in automated fashion (e.g. by a software application). Automated primer selection for multiplex PCR may be accomplished employing a software program designed as shown by the flow chart in FIG. 17.

Multiplex PCR commonly requires extensive optimization to avoid biased amplification of select amplicons and the amplification of spurious products resulting from the formation of primer-dimers. In order to avoid these problems, the present invention provides methods and software application that provide selection criteria to generate a primer set configured for multiplex PCR, and subsequent use in a detection assay (e.g. INVADER detection assays).

In some embodiments, the methods and software applications of the present invention start with user defined sequences and corresponding target sequence locations. In certain embodiments, the methods and/or software application determines a footprint region within the target sequence (the minimal amplicon required for INVADER detection) for each sequence. The footprint region includes the region where assay probes hybridize, as well as any user defined additional bases extending outward therefore (e.g. 5 additional bases included on each side of where the assay probes hybridize). Next, primers are designed outward from the footprint region and evaluated against several criteria, including the potential for primer-dimer formation with previously designed primers in the current multiplexing set. This process may be continued, through multiple iterations of the same set of sequences until primers against all sequences in the current multiplexing set can be designed.

Once a primer set is designed for multiplex PCR, this set may be employed, in some embodiments. Multiplex PCR may be carried out, for example, under standard conditions using only 10 ng of genomic DNA as template. After 10 min at 95° C., Taq (2.5 units) may be added to a 50 ul reaction and PCR carried out for 50 cycles. The PCR reaction may be diluted and loaded directly onto an solid support format (3 ul/well) (See FIG. 16). An additional 3 ul of 15 mM MgCl₂ may be added to each reaction on the plate and covered with 6 ul of mineral oil. The entire plate may then be heated to 95° C. for 5 min. and incubated at 63° C. for 40 min. FAM and RED fluorescence may then be measured on a Cytofluor 4000 fluorescent plate reader and “Fold Over Zero” (FOZ) values calculated for each amplicon. Results from each target may be color coded in a table as “pass” (green), “mis-call” (pink), or “no-call” (white).

In some embodiments the number of PCR reactions is from about 1 to about 10 reactions. In some embodiments, the number of PCR reactions is from about 10 to about 50 reactions. In further embodiments, the number of PCR reactions is from about 50 to about 100. In additional embodiments, the number of PCR reactions is greater than 100.

The present invention also provides methods to optimize multiplex PCR reactions (e.g. once a primer set is generated, the concentration of each primer or primer pair may be optimized). For example, once a primer set has been generated and used in a multiplex PCR at equal molar concentrations, the primers may be evaluated separately such that the optimum primer concentration is determined such that the multiplex primer set performs better.

Multiplex PCR reactions are being recognized in the scientific, research, clinical and biotechnology industries as potentially time effective and less expensive means of obtaining nucleic acid information compared to standard, monoplex PCR reactions. Instead of performing only a single amplification reaction per reaction vessel (tube or well of a multi-well plate for example), numerous amplification reactions are performed in a single reaction vessel.

The cost per target is theoretically lowered by eliminating technician time in assay set-up and data analysis, and by the substantial reagent savings (especially enzyme cost). Another benefit of the multiplex approach is that far less target sample is required.

To design primers for a successful multiplex PCR reaction, the issue of aberrant interaction among primers should be addressed. The formation of primer dimers, even if only a few bases in length, may inhibit both primers from correctly hybridizing to the target sequence. Further, if the dimers form at or near the 3′ ends of the primers, no amplification or very low levels of amplification will occur, since the 3′ end is required for the priming event. Clearly, the more primers utilized per multiplex reaction, the more aberrant primer interactions are possible. The methods, systems and applications of the present help prevent primer dimers in large sets of primers, making the set suitable for highly multiplexed PCR.

When designing primer pairs for numerous sites (for example 100 sites in a multiplex PCR reaction), the order in which primer pairs are designed can influence the total number of compatible primer pairs for a reaction. For example, if a first set of primers is designed for a first target region that happens to be an A/T rich target region, these primers will be A/T rich. If the second target region chosen also happens to be an A/T rich target region, it is far more likely that the primers designed for these two sets will be incompatible due to aberrant interactions, such as primer dimers. If, however, the second target region chosen is not A/T rich, it is much more likely that a primer set can be designed that will not interact with the first A/T rich set. For any given set of input target sequences, the present invention randomizes the order in which primer sets are designed. Furthermore, in some embodiments, the present invention re-orders the set of input target sequences in a plurality of different, random orders to maximize the number of compatible primer sets for any given multiplex reaction. In certain embodiments, the primers are designed such that GC-rich and AT-rich regions are avoided.

The present invention provides criteria for primer design that minimizes 3′ interactions (e.g. 3′ complementarity of primers is avoided to reduce probability of primer-dimer formation), while maximizing the number of compatible primer pairs for a given set of reaction targets in a multiplex design. For primers described as 5′-N[x]-N[x−1]- . . . -N[4]-N[3]-N[2]-N[1]-3′, N[1] is an A or C (in alternative embodiments, N[1] is a G or T). N[2]-N[1] of each of the forward and reverse primers designed should not be complementary to N[2]-N[1] of any other oligonucleotide. In certain embodiments, N[3]-N[2]-N[1] should not be complementary to N[3]-N[2]-N[1] of any other oligonucleotide. In preferred embodiments, if these criteria are not met at a given N[1], the next base in the 5′ direction for the forward primer or the next base in the 3′ direction for the reverse primer may be evaluated as an N[1] site. This process is repeated, in conjunction with the target randomization, until all criteria are met for all, or a large majority of, the targets sequences (e.g. 95% of target sequences can have primer pairs made for the primer set that fulfill these criteria).

Another challenge to be overcome in a multiplex primer design is the balance between actual, required nucleotide sequence, sequence length, and the oligonucleotide melting temperature (Tm) constraints. Importantly, since the primers in a multiplex primer set in a reaction should function under the same reaction conditions of buffer, salts and temperature, they need therefore to have substantially similar Tm's, regardless of GC or AT richness of the region of interest. The present invention allows for primer design that meets minimum Tm and maximum Tm requirements and minimum and maximum length requirements. For example, in the formula for each primer 5′-N[x]-N[x−1]- . . . -N[4]-N[3]-N[2]-N[1]-3′, x is selected such the primer has a predetermined melting temperature (e.g. bases are included in the primer until the primer has a calculated melting temperature of about 50 degrees Celsius). In certain embodiments, each of the primers in a set has the same melting temperature.

Often the products of a PCR reaction are used as the target material for another nucleic acid detection means, such as a hybridization-type detection assays, or the INVADER reaction assays for example. Consideration should be given to the location of primer placement to allow for the secondary reaction to successfully occur, and again, aberrant interactions between amplification primers and secondary reaction oligonucleotides should be minimized for accurate results and data. Selection criteria may be employed such that the primers designed for a multiplex primer set do not react (e.g. hybridize with, or trigger reactions) with oligonucleotide components of a detection assay. For example, in order to prevent primers from reacting with the FRET oligonucleotide of a bi-plex INVADER assay, certain homology criteria is employed. In particular, if each of the primers in the set are defined as 5′-N[x]-N[x−1]- . . . -N[4]-N[3]-N[2]-N[1]-3′, then N[4]-N[3]-N[2]-N[l]-3′ is selected such that it is less than 90% homologous with the FRET or INVADER oligonucleotides. In other embodiments, N[4]-N[3]-N[2]-N[1]-3′ is selected for each primer such that it is less than 80% homologous with the FRET or INVADER oligonucleotides. In certain embodiments, N[4]-N[3]-N[2]-N[1]-3′ is selected for each primer such that it is less than 70% homologous with the FRET or INVADER oligonucleotides.

While employing the criteria of the present invention to develop a primer set, some primer pairs may not meet all of the stated criteria (these may be rejected as errors). For example, in a set of 100 targets, 30 are designed and meet all listed criteria, however, set 31 fails. In the method of the present invention, set 31 may be flagged as failing, and the method could continue through the list of 100 targets, again flagging those sets that do not meet the criteria. Once all 100 targets have had a chance at primer design, the method would note the number of failed sets, re-order the 100 targets in a new random order and repeat the design process. After a configurable number of runs, the set with the most passed primer pairs (the least number of failed sets) are chosen for the multiplex PCR reaction.

Target sequences and/or primer pairs are entered into the system. The first set of boxes show how target sequences are added to the list of sequences that have a footprint determined, while other sequences are passed immediately into the primer set pool (e.g. PDPass, those sequences that have been previously processed and shown to work together without forming Primer dimers or having reactivity to FRET sequences), as well as DimerTest entries (e.g. pair or primers a user wants to use, but that has not been tested yet for primer dimer or fret reactivity). In other words, the initial set of boxes leading up to “end of input” sort the sequences so they can be later processed properly.

The primer pool is basically cleared or “emptied” to start a fresh run. The target sequences are then sent to “B” to be processed, and DimerTest pairs are sent to “C” to be processed. Target sequences are sent to “B”, where a user or software application determines the footprint region for the target sequence (e.g. where the assay probes will hybridize in order to detect the target sequence). It is important to design this region (which the user may further expand by defining that additional bases past the hybridization region be added) such that the primers that are designed fully encompass this region. In some embodiments, the software application INVADER CREATOR is used to design the INVADER oligonucleotide and downstream probes that will hybridize with the target region (although any type of program of system could be used to create any type of probes a user was interested in designing probes for, and thus determining the footprint region for on the target sequence). Thus the core footprint region is then defined by the location of these two assay probes on the target.

Next, the system starts from the 5′ edge of the footprint and travels in the 5′ direction until the first base is reached, or until the first A or C (or G or T) is reached. This is set as the initial starting point for defining the sequence of the forward primer (i.e. this serves as the initial N[1] site). From this initial N[1] site, the sequence of the primer for the forward primer is the same as those bases encountered on the target region. For example, if the default size of the primer is set as 12 bases, the system starts with the bases selected as N[1] and then adds the next 11 bases found in the target sequences. This 12-mer primer is then tested for a melting temperature (e.g. using INVADER CREATOR), and additional bases are added from the target sequence until the sequence has a melting temperature that is designated by the user (e.g. about 50 degrees Celsius, and not more than 55 degrees Celsius). For example, the system employs the formula 5′-N[x]-N[x−1]- . . . -N[4]-N[3]-N[2]-N[1]-3′, and x is initially 12. Then the system adjusts x to a higher number (e.g. longer sequences) until the pre-set melting temperature is found.

The next step is to determine if the primer that has been designed so far will cause primer-dimer and/or fret reactivity (e.g. with the other sequences already in the pool). The criteria used for this determination are explained above. If the primer passes this step, the forward primer is added to the primer pool. However, if the forward primer fails this criteria, the starting point (N[1] is moved) one nucleotide in the 5′ direction (or to the next A or C, or next G or T). The system first checks to make sure shifting over leaves enough room on the target sequence to successfully make a primer. If yes, the system loops back and check this new primer for melting temperature. However, if no sequence can be designed, then the target sequence is flagged as an error (e.g. indicating that no forward primer can be made for this target).

This same process is then repeated for designing the reverse primer. If a reverse primer is successfully made, then the pair or primers is put into the primer pool, and the system goes back to “B” (if there are more target sequences to process), or goes onto “C” to test DimerTest pairs.

If there are no DimerTest pairs, the system goes on to “D”. However, if there are DimerTest pairs, these are tested for primer-dimer and/or FRET reactivity as described above. If the DimerTest pair fails these criteria they are flagged as errors. If the DimerTest pair passes the criteria, they are added to the primer set pool, and then the system goes back to “C” if there are more DimerTest pairs to be evaluated, or goes on to “D” if there are no more DimerTest pairs to be evaluated.

Starting at “D”, the pool of primers that has been created is evaluated. The first step in this section is to examine the number of error (failures) generated by this particular randomized run of sequences. If there were no errors, this set is the best set as maybe outputted to a user. If there are more than zero errors, the system compares this run to any other previous runs to see what run resulted in the fewest errors. If the current run has fewer errors, it is designated as the current best set. At this point, the system may go back to “A” to start the run over with another randomized set of the same sequences, or the pre-set maximum number of runs (e.g. 5 runs) may have been reached on this run (e.g. this was the 5th run, and the maximum number of runs was set as 5). If the maximum has been reached, then the best set is outputted as the best set. This best set of primers may then be used to generate as physical set of oligonucleotides such that a multiplex PCR reaction may be carried out.

Another challenge to be overcome with multiplex PCR reactions is the unequal amplicon concentrations that result in a standard multiplex reaction. The different loci targeted for amplification may each behave differently in the amplification reaction, yielding vastly different concentrations of each of the different amplicon products. The present invention provides methods, systems, software applications, computer systems, and a computer data storage medium that may be used to adjust primer concentrations relative to a first detection assay read (e.g. INVADER assay read), and then with balanced primer concentrations come close to substantially equal concentrations of different amplicons.

The concentrations for various primer pairs may be determined experimentally. In some embodiments, there is a first run conducted with all of the primers in equimolar concentrations. Time reads are then conducted. Based upon the time reads, the relative amplification factors for each amplicon are determined. Then based upon a unifying correction equation, an estimate of what the primer concentration should be obtained to get the signals closer within the same time point. These detection assays can be on an array of different sizes (384 well plates).

It is appreciated that combining the invention with detection assays and arrays of detection assays provides substantial processing efficiencies. Employing a balanced mix of primers or primer pairs created using the invention, a single point read can be carried out so that an average user can obtain great efficiencies in conducting tests that require high sensitivity and specificity across an array of different targets.

Having optimized primer pair concentrations in a single reaction vessel allows the user to conduct amplification for a plurality or multiplicity of amplification targets in a single reaction vessel and in a single step. The yield of the single step process is then used to successfully obtain test result data for, for example, several hundred assays. For example, each well on a 384 well plate can have a different detection assay thereon. The results of the single step mutliplex PCR reaction has amplified 384 different targets of genomic DNA, and provides you with 384 test results for each plate. Where each well has a plurality of assays even greater efficiencies can be obtained.

Therefore, the present invention provides the use of the concentration of each primer set in highly multiplexed PCR as a parameter to achieve an unbiased amplification of each PCR product. Any PCR includes primer annealing and primer extension steps. Under standard PCR conditions, high concentration of primers in the order of 1 uM ensures fast kinetics of primers annealing while the optimal time of the primer extension step depends on the size of the amplified product and can be much longer than the annealing step. By reducing primer concentration, the primer annealing kinetics can become a rate limiting step and PCR amplification factor should strongly depend on primer concentration, association rate constant of the primers, and the annealing time.

The binding of primer P with target T can be described by the following model:

$$P + T {\to }^{ {k}_{a }} PT \qquad ( 1 )$$

where kₐ is the association rate constant of primer annealing. We assume that the annealing occurs at the temperatures below primer melting and the reverse reaction can be ignored.

The solution for this kinetics under the conditions of a primer excess is well known:

[PT]=T₀(1−e^{-k}^{ₐ}^{ct}) (2)

where [PT] is the concentration of target molecules associated with primer, T₀ is initial target concentration, c is the initial primer concentration, and t is primer annealing time. Assuming that each target molecule associated with primer is replicated to produce full size PCR product, the target amplification factor in a single PCR cycle is

$$Z = \frac{{T}_{0} + [ PT ]}{{T}_{0}} = 2 - {ⅇ}^{- {k}_{a} ct} \qquad ( 3 )$$

The total PCR amplification factor after n cycles is given by

F=Zⁿ=(2−e^{-k}^{ₐ}^{ct})ⁿ (4)

As it follows from equation 4, under the conditions where the primer annealing kinetics is the rate limiting step of PCR, the amplification factor should strongly depend on primer concentration. Thus, biased loci amplification, whether it is caused by individual association rate constants, primer extension steps or any other factors, can be corrected by adjusting primer concentration for each primer set in the multiplex PCR. The adjusted primer concentrations can be also used to correct biased performance of INVADER assay used for analysis of PCR pre-amplified loci. Employing this basic principle, the present invention has demonstrated a linear relationship between amplification efficiency and primer concentration and used this equation to balance primer concentrations of different amplicons, resulting in the equal amplification of ten different amplicons in PCR Primer Design Example 1. This technique may be employed on any size set of multiplex primer pairs. In some embodiments, the PCR primers are unoptimized, and the INVADER assay is employed to detect the amplified products (See, Ohnishi et al., J. Hum. Genet. 46:471-7, 2001, herein incorporated by reference.

i. PCR Primer Design Example 1

The following experimental example describes the manual design of amplification primers for a multiplex amplification reaction, and the subsequent detection of the amplicons by the INVADER assay.

Ten target sequences were selected from a set of pre-validated SNP-containing sequences, available in a TWT in-house oligonucleotide order entry database. Each target contains a single nucleotide polymorphism (SNP) to which an INVADER assay had been previously designed. The INVADER assay oligonucleotides were designed by the INVADER CREATOR software (Third Wave Technologies, Inc. Madison, Wis.), thus the footprint region in this example is defined as the INVADER “footprint”, or the bases covered by the INVADER and the probe oligonucleotides, optimally positioned for the detection of the base of interest, in this case, a single nucleotide polymorphism. About 200 nucleotides of each of the 10 target sequences were analyzed for the amplification primer design analysis, with the SNP base residing about in the center of the sequence.

Criteria of maximum and minimum probe length (defaults of 30 nucleotides and 12 nucleotides, respectively) were defined, as was a range for the probe melting temperature Tm of 50-60° C. In this example, to select a probe sequence that will perform optimally at a pre-selected reaction temperature, the melting temperature (Tₘ) of the oligonucleotide is calculated using the nearest-neighbor model and published parameters for DNA duplex formation (Allawi and SantaLucia, Biochemistry, 36:10581 [1997], herein incorporated by reference). Because the assay's salt concentrations are often different than the solution conditions in which the nearest-neighbor parameters were obtained (1M NaCl and no divalent metals), and because the presence and concentration of the enzyme influence optimal reaction temperature, an adjustment should be made to the calculated Tₘ to determine the optimal temperature at which to perform a reaction. One way of compensating for these factors is to vary the value provided for the salt concentration within the melting temperature calculations. This adjustment is termed a ‘salt correction’. The term “salt correction” refers to a variation made in the value provided for a salt concentration for the purpose of reflecting the effect on a Tₘ calculation for a nucleic acid duplex of a non-salt parameter or condition affecting said duplex. Variation of the values provided for the strand concentrations will also affect the outcome of these calculations. By using a value of 280 nM NaCl (SantaLucia, Proc Natl Acad Sci USA, 95:1460 [1998], herein incorporated by reference) and strand concentrations of about 10 pM of the probe and 1 fM target, the algorithm for used for calculating probe-target melting temperature has been adapted for use in predicting optimal primer design sequences.

Next, the sequence adjacent to the footprint region, both upstream and downstream were scanned and the first A or C was chosen for design start such that for primers described as 5′-N[x]-N[x−1]- . . . -N[4]-N[3]-N[2]-N[1]-3′, where N[1] should be an A or C. Primer complementarity was avoided by using the rule that: N[2]-N[1] of a given oligonucleotide primer should not be complementary to N[2]-N[1] of any other oligonucleotide, and N[3]-N[2]-N[1]should not be complementary to N[3]-N[2]-N[1] of any other oligonucleotide. If these criteria were not met at a given N[1], the next base in the 5′ direction for the forward primer or the next base in the 3′ direction for the reverse primer will be evaluated as an N[1] site. In the case of manual analysis, A/C rich regions were targeted in order to minimize the complementarity of 3′ ends.

In this example, an INVADER assay was performed following the multiplex amplification reaction. Therefore, a section of the secondary INVADER reaction oligonucleotide (the FRET oligonucleotide sequence) was also incorporated as criteria for primer design; the amplification primer sequence should be less than 80% homologous to the specified region of the FRET oligonucleotide.

All primers were synthesized according to standard oligonucleotide chemistry, desalted (by standard methods) and quantified by absorbance at A260 and diluted to 50 μM concentrated stock. Multiplex PCR was then carried out using 10-plex PCR using equimolar amounts of primer (0.01 uM/primer) under the following conditions; 100 mM KCl, 3 mM MgCl, 10 mM Tris pH8.0, 200 uM dNTPs, 2.5U taq, and 10 ng of human genomic DNA (hgDNA) template in a 50 ul reaction. The reaction was incubated for (94C/30 sec, 50C/44 sec.) for 30 cycles. After incubation, the multiplex PCR reaction was diluted 1:10 with water and subjected to INVADER analysis using INVADER Assay FRET Detection Plates, 96 well genomic biplex, 100 ng CLEAVASE VIII, INVADER assays were assembled as 15 ul reactions as follows; 1 ul of the 1:10 dilution of the PCR reaction, 3 ul of PPI mix, 5 ul of 22.5 mM MgCl2, 6 ul of dH20, covered with 15 ul of Chillout. Samples were denatured in the INVADER biplex by incubation at 95C for 5 min., followed by incubation at 63C and fluorescence measured on a Cytofluor 4000 at various timepoints.

Using the following criteria to accurately make genotyping calls (FOZ_FAM+FOZ_RED-2>0.6), only 2 of the 10 INVADER assay calls can be made after 10 minutes of incubation at 63C, and only 5 of the 10 calls could be made following an additional 50 min of incubation at 63C (60 min.). At the 60 min time point, the variation between the detectable FOZ values is over 100 fold between the strongest signal (41646, FAM_FOZ+RED_FOZ-2=54.2, which is also is far outside of the dynamic range of the reader) and the weakest signal (67356, FAM_FOZ+RED_FOZ-2=0.2). Using the same INVADER assays directly against 100 ng of human genomic DNA (where equimolar amounts of each target would be available), all reads could be made with in the dynamic range of the reader and variation in the FOZ values was approximately seven fold between the strongest (53530, FAM_FOZ+RED_FOZ-2=3.1) and weakest (53530, FAM_FOZ+RED_FOZ-2=0.43) of the assays. This suggests that the dramatic discrepancies in FOZ values seen between different amplicons in the same multiplex PCR reaction is a function of biased amplification, and not variability attributable to INVADER assay. Under these conditions, FOZ values generated by different INVADER assays are directly comparable to one another and can reliably be used as indicators of the efficiency of amplification.

Estimation of amplification factor of a given amplicon using FOZ values. In order to estimate the amplification factor (F) of a given amplicon, the FOZ values of the INVADER assay can be used to estimate amplicon abundance. The FOZ of a given amplicon with unknown concentration at a given time (FOZm) can be directly compared to the FOZ of a known amount of target (e.g. 100 ng of genomic DNA=30,000 copies of a single gene) at a defined point in time (FOZ₂₄₀, 240 min) and used to calculate the number of copies of the unknown amplicon. In equation 1, FOZm represents the sum of RED_FOZ and FAM_FOZ of an unknown concentration of target incubated in an INVADER assay for a given amount of time (m). FOZ₂₄₀ represents an empirically determined value of RED_FOZ (using INVADER assay 41646), using for a known number of copies of target (e.g. 10 ng of hgDNA≈30,000 copies) at 240 minutes.

F=((FOZₘ−1)*500/(FOZ₂₄₀−1))*(240/m)\hat{ }2 (equation 1a)

Although equation 1a is used to determine the linear relationship between primer concentration and amplification factor F, equation 1a′ is used in the calculation of the amplification factor F for the 10-plex PCR (both with equimolar amounts of primer and optimized concentrations of primer), with the value of D representing the dilution factor of the PCR reaction. In the case of a 1:3 dilution of the 50 ul multiplex PCR reaction. D=0.3333.

F=((FOZₘ−2)*500/(FOZ₂₄₀−1)*D)*(240/m)\hat{ }2 (equation 1a′)

Although equations 1a and 1a′ will be used in the description of the 10-plex multiplex PCR, a more correct adaptation of this equation was used in the optimization of primer concentrations in the 107 plex PCR. In this case, FOZ₂₄₀=the average of FAM_FOZ₂₄₀+RED_FOZ₂₄₀ over the entire INVADER MAP plate using hgDNA as target (FOZ₂₄₀=3.42) and the dilution factor D is set to 0.125.

F=((FOZₘ−2)*500/(FOZ₂₄₀−2)*D)*(240/m)\hat{ }2 (equation 1b)

It should be noted that in order for the estimation of amplification factor F to be more accurate, FOZ values should be within the dynamic range of the instrument on which the reading are taken. In the case of the Cytofluor 4000 used in this study, the dynamic range was between about 1.5 and about 12 FOZ.

Section 3. Linear Relationship Between Amplification Factor and Primer Concentration.

In order to determine the relationship between primer concentration and amplification factor (F), four distinct uniplex PCR reactions were run at using primers 1117-70-17 and 1117-70-18 at concentrations of 0.01 uM, 0.012 uM, 0.014 uM, 0.020 uM respectively. The four independent PCR reactions were carried out under the following conditions; 100 mM KCl, 3 mM MgCl, 10 mM Tris pH 8.0, 200 uM dNTPs using 10 ng of hgDNA as template. Incubation was carried out at (94C/30 sec., 50C/20 sec.) for 30 cycles. Following PCR, reactions were diluted 1:10 with water and run under standard conditions using INVADER Assay FRET Detection Plates, 96 well genomic biplex, 100 ng CLEAVASE VIII enzyme. Each 15 ul reaction was set up as follows; 1 ul of 1:10 diluted PCR reaction, 3 ul of the PPI mix SNP#47932, 5 ul 22.5 mM MgCl2, 6 ul of water, 15 ul of Chillout. The entire plate was incubated at 95C for 5 min, and then at 63C for 60 min at which point a single read was taken on a Cytofluor 4000 fluorescent plate reader. For each of the four different primer concentrations (0.01 uM, 0.012 uM, 0.014 uM, 0.020 uM) the amplification factor F was calculated using equation 1a, with FOZm=the sum of FOZ_FAM and FOZ_RED at 60 minutes, m=60, and FOZ₂₄₀=1.7. In plotting the primer concentration of each reaction against the log of the amplification factor Log(F), a strong linear relationship was noted (FIG. 20). Using the data points in FIG. 20, the formula describing the linear relationship between amplification factor and primer concentration is described in equation 2:

Y=1.684X+2.6837 (equation 2a)

Using equation 2, the amplification factor of a given amplicon Log(F)=Y could be manipulated in a predictable fashion using a known concentration of primer (X). In a converse manner, amplification bias observed under conditions of equimolar primer concentrations in multiplex PCR, could be measured as the “apparent” primer concentration (X) based on the amplification factor F. In multiplex PCR, values of “apparent” primer concentration among different amplicons can be used to estimate the amount of primer of each amplicon required to equalize amplification of different loci:

X=(Y−2.6837)/1.68 (equation 2b)

Section 4.Calculation of Apparent Primer Concentrations from a Balanced Multiplex Mix.

As described in a previous section, primer concentration can directly influence the amplification factor of given amplicon. Under conditions of equimolar amounts of primers, FOZm readings can be used to calculate the “apparent” primer concentration of each amplicon using equation 2. Replacing Y in equation 2 with log(F) of a given amplification factor and solving for X, gives an “apparent” primer concentration based on the relative abundance of a given amplicon in a multiplex reaction. Using equation 2 to calculate the “apparent” primer concentration of all primers (provided in equimolar concentration) in a multiplex reaction, provides a means of normalizing primer sets against each other. In order to derive the relative amounts of each primer that should be added to an “Optimized” multiplex primer mix R, each of the “apparent” primer concentrations should be divided into the maximum apparent primer concentration (Xₘₐₓ), such that the strongest amplicon is set to a value of 1 and the remaining amplicons to values equal or greater than 1

R[n]=Xmax/X[n] (equation 3)

Using the values of R[n] as an arbitrary value of relative primer concentration, the values of R[n] are multiplied by a constant primer concentration to provide working concentrations for each primer in a given multiplex reaction. In the example shown, the amplicon corresponding to SNP assay 41646 has an R[n] value equal to 1. All of the R[n] values were multiplied by 0.01 uM (the original starting primer concentration in the equimolar multiplex pcr reaction) such that lowest primer concentration is R[n] of 41646 which is set to 1, or 0.01 uM. The remainder of the primer sets were also proportionally increased. The results of multiplex PCR with the “optimized” primer mix are described below.

Section 5 Using Optimized Primer Concentrations in Multiplex PCR, Variation in FOZ's among 10 INVADER assays are greatly reduced.

Multiplex PCR was carried out using 10-plex PCR using varying amounts of primer based on the volumes indicated (X[max] was SNP41646, setting 1x=0.01 uM/primer). Multiplex PCR was carried out under conditions identical to those used in with equimolar primer mix; 100 mMKCl, 3 mMMgCl, 10 mM Tris pH8.0, 200 uM dNTPs, 2.5U taq, and 10 ng of hgDNA template in a 50 ul reaction. The reaction was incubated for (94C/30 sec, 50C/44 sec.) for 30 cycles. After incubation, the multiplex PCR reaction was diluted 1:10 with water and subjected to INVADER analysis. Using INVADER Assay FRET Detection Plates, (96 well genomic biplex, 100 ng CLEAVASE VIII enzyme), reactions were assembled as 15 ul reactions as follows; 1 ul of the 1:10 dilution of the PCR reaction, 3 ul of the appropriate PPI mix, 5 ul of 22.5 mM MgCl2, 6 ul of dH20. An additional 15 ul of CHILL OUT was added to each well, followed by incubation at 95C for 5 min. Plates were incubated at 63C and fluorescence measured on a Cytofluor 4000 at 10 min.

Using the following criteria to accurately make genotyping calls (FOZ_FAM+FOZ_RED-2>0.6), all 10 of 10 (100%) INVADER calls can be made after 10 minutes of incubation at 63C. In addition, the values of FAM+RED-2 (an indicator of overall signal generation, directly related to amplification factor (see equation 2)) varied by less than seven fold between the lowest signal (67325, FAM+RED-2=0.7) and the highest (FIG. 22, 47892, FAM+RED-2=4.3).

ii. PCR Primer Design Example 2

Using the TWT Oligo Order Entry Database, 144 sequences of less than 200 nucleotides in length were obtained with SNP annotated using brackets to indicate the SNP position for each sequence (e.g. NNNNNNN[N_{(wt)}/N₍ₘₜ₎]NNNNNNNN). In order to expand sequence data flanking the SNP of interest, sequences were expanded to approximately 1 kB in length (500 nts flanking each side of the SNP) using BLAST analysis. Of the 144 starting sequences, 16 could not expanded by BLAST, resulting in a final set of 128 sequences expanded to approximately 1 kB length. These expanded sequences were provided to the user in Excel format with the following information for each sequence; (1) TWT Number, (2) Short Name Identifier, and (3) sequence. The Excel file was converted to a comma delimited format and used as the input file for Primer Designer INVADER CREATOR v1.3.3. software (this version of the program does not screen for FRET reactivity of the primers, nor does it allow the user to specify the maximum length of the primer). INVADER CREATOR Primer Designer v1.3.3., was run using default conditions (e.g. minimum primer size of 12, maximum of 30), with the exception of Tm_{low} which was set to 60C. The output file contained 128 primer sets (256 primers, See FIG. 25), four of which were thrown out due to excessively long primer sequences (SNP #47854, 47889, 54874, 67396), leaving 124 primers sets (248 primers) available for synthesis. The remaining primers were synthesized using standard procedures at the 200 nmol scale and purified by desalting. After synthesis failures, 107 primer sets were available for assembly of an equimolar 107-plex primer mix (214 primers, See FIG. 25). Of the 107 primer sets available for amplification, only 101 were present on the INVADER MAP plate to evaluate amplification factor.

Multiplex PCR was carried out using 101-plex PCR using equimolar amounts of primer (0.025 uM/primer) under the following conditions; 100 mMKCl, 3 mM MgCl, 10 mM Tris pH8.0, 200 uM dNTPs, and 10 ng of human genomic DNA (hgDNA) template in a 50 ul reaction. After denaturation at 95C for 10 min, 2.5 units of Taq was added and the reaction incubated for (94C/30 sec, 50C/44 sec.) for 50 cycles. After incubation, the multiplex PCR reaction was diluted 1:24 with water and subjected to INVADER assay analysis using INVADER MAP detection platform. Each INVADER MAP assay was run as a 6 ul reaction as follows; 3 ul of the 1:24 dilution of the PCR reaction (total dilution 1:8 equaling D=0.125), 3 ul of 15 mM MgCl2 covered with covered with 6 ul of CHILLOUT. Samples were denatured in the INVADER MAP plate by incubation at 95C for 5 min., followed by incubation at 63C and fluorescence measured on a Cytofluor 4000 (384 well reader) at various timepoints over 160 minutes. Analysis of the FOZ values calculated at 10, 20, 40, 80, 160 min. shows that correct calls (compared to genomic calls of the same DNA sample) could be made for 94 of the 101 amplicons detectable by the INVADER MAP platform (FIG. 26 and FIG. 27). This provides proof that the INVADER CREATOR Primer Designer software can create primer sets which function in highly multiplex PCR.

In using the FOZ values obtained throughout the 160 min. time course, amplification factor F and R[n] were calculated for each of the 101 amplicons. R[nmax] was set at 1.6, which although Low end corrections were made for amplicons which failed to provide sufficient FOZm signal at 160 min., assigning an arbitrary value of 12 for R[n]. High end corrections for amplicons whose FOZm values at the 10 min. read, an R[n] value of 1 was arbitrarily assigned. Optimized primer concentrations of the 101-plex were calculated using the basic principles outlined in the 10-plex example and equation 1b, with an R[n] of 1 corresponding to 0.025 uM primer (see FIG. 15 for various primer concentrations). Multiplex PCR was under the following conditions; 100 mMKCl, 3 mM MgCl, 10 mM Tris pH8.0, 200 uM dNTPs, and 10 ng of human genomic DNA (hgDNA) template in a 50 ul reaction. After denaturation at 95C for 10 min, 2.5 units of Taq was added and the reaction incubated for (94C/30 sec, 50C/44 sec.) for 50 cycles. After incubation, the multiplex PCR reaction was diluted 1:24 with water and subjected to INVADER analysis using INVADER MAP detection platform. Each INVADER MAP assay was run as a 6 ul reaction as follows; 3 ul of the 1:24 dilution of the PCR reaction (total dilution 1:8 equaling D=0.125), 3 ul of 15 mM MgCl2 covered with covered with 6 ul of CHILLOUT. Samples were denatured in the INVADER MAP plate by incubation at 95C for 5 min., followed by incubation at 63C and fluorescence measured on a Cytofluor 4000 (384 well reader) at various timepoints over 160 minutes. Analysis of the FOZ values was carried out at 10, 20, and 40 min. and compared to calls made directly against the genomic DNA. Shown in FIG. 26, is a comparison between calls made at 10 min. with a 101-plex PCR with the equimolar primer concentrations versus calls that were made at 10 min. with a 101-plex PCR run under optimized primer concentrations. Under equimolar primer concentration, multiplex PCR results in only 50 correct calls at the 10 min time point, where under optimized primer concentrations multiplex PCR results in 71 correct calls, resulting in a gain of 21 (42%) new calls. Although all 101 calls could not be made at the 10 min timepoint, 94 calls could be made at the 40 min. timepoint suggesting the amplification efficiency of the majority of amplicons had improved. Unlike the 10-plex optimization that only required a single round of optimization, multiple rounds of optimization may be required for more complex multiplexing reactions to balance the amplification of all loci.

B. Other Detection Assays

The present invention is not limited to the INVADER assay. Any suitable nucleic acid detection assay may be utilized including, but not limited to, those disclosed below.

1. Direct Sequencing Assays

In some embodiments of the present invention, nucleic acid sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the nucleic acid sequence of interest) is sequenced using any suitable method, including but not limited to manual sequencing using radioactive marker nucleotides, or automated sequencing. The results of the sequencing are displayed using any suitable method. The sequence is examined and the presence or absence of a given target is determined.

2. PCR Assay

In some embodiments of the present invention, nucleic acid sequences are detected using a PCR-based assay. In some embodiments, the PCR assay comprises the use of oligonucleotide primers that hybridize only to the desired nucleic acid sequence. Both sets of primers are used to amplify a sample of DNA.

3. Fragment Length Polymorphism Assays

In some embodiments of the present invention, nucleic acid sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a target sequence will have a different banding pattern than a control lacking the sequence.

a. RFLP Assay

In some embodiments of the present invention, nucleic acid sequences are detected using a restriction fragment length polymorphism assay (RFLP). The region of interest is first isolated using PCR. The PCR products are then cleaved with restriction enzymes known to give a unique length fragment for a given nucleic acid sequence. The restriction-enzyme digested PCR products are generally separated by gel electrophoresis and may be visualized by ethidium bromide staining. The length of the fragments is compared to molecular weight markers and fragments generated from controls.

b. CFLP Assay

In other embodiments, nucleic acid sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. In preferred embodiments, one or both strands are labeled. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by denaturing gel electrophoresis) and visualized (e.g., by autoradiography, fluorescence imaging or staining). The length of the fragments is compared to molecular weight markers and fragments generated from controls.

4. Hybridization Assays

In preferred embodiments of the present invention, nucleic acid sequences are detected a hybridization assay. In a hybridization assay, the presence of absence of a given SNP or nucleic acid sequence is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

a. Direct Detection of Hybridization

In some embodiments, hybridization of a probe to the sequence of interest (e.g., a SNP or mutation) is detected directly by visualizing a bound probe (e.g., a Northern or Southern assay; See e.g., Ausabel et al. (eds.), Current Protocols in Molecular Biology, John Wiley & Sons, NY [1991]). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labeled probe.

b. Detection of Hybridization Using “DNA Chip” Assays

In some embodiments of the present invention, nucleic acid sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding,

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the targets of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

C. Enzymatic Detection of Hybridization

In some embodiments of the present invention, hybridization is detected by enzymatic cleavage of specific structures. In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of DNA polymerases such as AMPLITAQ DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, target sequences are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labeled antibody specific for biotin).

5. Other Detection Assays

Additional detection assays that are suitable for use in the systems and methods of the present invention include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884 and 6,183,960, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay Proc. Natl. Acad. Sci USA 88, 189-93 (1991)); and sandwich hybridization methods (e.g., U.S. Pat. No. 5,288,609, herein incorporated by reference in its entirety).

6. Mass Spectroscopy Assay

In some embodiments, a MassARRAY system (Sequenom, San Diego, Calif.) is used to detect nucleic acid sequences (See e.g., U.S. Pat. Nos. 6,043,031; 5,777,324; and 5,605,798; each of which is herein incorporated by reference). DNA is isolated from blood samples using standard procedures. Next, specific DNA regions containing the nucleic acid sequence of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization—Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

In some embodiments, data generated by different detection methods are processed to facilitate comparison, e.g., using an process like the Extraction-Transformation-Load paradigm from Data Warehousing, wherein data is “published” into a single repository, normalizing disparate data, and optimizing it for browsing and easy access to normalized, integrated data (e.g., DataMart and MetaSymphony software, NetGenics, Inc., Cleveland Ohio; U.S. Pat. No. 6,125,383, incorporated herein by reference in its entirety). SNP data generated by one SNP analysis method may be compared to results data generated by another analysis method (e.g., INVADER assay results are compared to gene chip data).

In some embodiments of the present invention, data is processed using an algorithm selected to determine an allele from the input assay data. The algorithm selected for processing data may be determined by the nature of the input assay data. The following provides an example of the application of an allele caller to an assay run in a microtiter plate (e.g., a 384-well plate).

The user enters information to identify the plate to be analyzed. In one embodiment, the plate may be identified by entry of a code number (e.g., a barcode number, part number, lot number). In another embodiment, the program provides a menu from which the user selects the number corresponding to the plate.

In some embodiments, the program provides a validation of the plate. For example, in some embodiments, the program verifies that the plate is of a suitable format for available analysis (e.g., that it corresponds to an assay for which an allele caller function can be provided). In other embodiments, the program verifies that the plate has been passed through some other process step. In some embodiments wherein the association database is provided on removable media (e.g., as described above), the program verifies that the version of the CD in use is suitable (e.g., has an appropriate version of an allele caller function, or has an appropriate association database) for use with the plate to be analyzed.

When a plate has been identified and determined to be valid for analysis, a record is displayed. In preferred embodiments, the record is a table having cells that correspond to assay wells on a microtiter plate (e.g., a “plate viewer”, described above). In some embodiments, the user has the option (e.g., through a menu selection) of creating a new analysis record or of calling up a record of a prior analysis. In preferred embodiments, the record links to identifying data from other analyses performed on the same collection of samples (e.g., name, date generated, etc.). In particularly preferred embodiments, SNP test wells on a plate are linked through a “plate viewer” function to SNP records in a database. In further particularly preferred embodiments, the database is an association database.

Prior to analysis, the assay data from the plate is imported, or “loaded” into the analysis program. It is contemplated that the data to be processed by an allele caller may be provided in many different forms. In some embodiments, the assay data is raw (i.e., unanalyzed) signal, such as a number corresponding to a measurement of fluorescence signal from a spot on a chip or a reaction vessel, or a number corresponding to measurement of a peak (e.g., peak height or area, as from, for example, a mass spectrometer, HPLC or capillary separation device). In some embodiments the data is imported directly from a measuring device. In other embodiments, the data is imported from a file. Raw assay data may be generated by any number of SNP detection methods, including but not limited to those listed above.

In some embodiments, the loaded assay data is displayed on a screen. In preferred embodiments, data is displayed in a plate viewer format. In some preferred embodiments, the layout is displayed in a new window. In particularly preferred embodiments, the window is printable.

Loaded assay data is then analyzed or processed using one or more algorithms selected to determine an allele from the input assay data. The algorithm selected for processing data is generally determined by the nature of the input assay data. In some embodiments, analysis involves determining the presence or absence of a signal (e.g., detectable fluorescence, or a detectable peak). In other embodiments, analysis involves determining the presence of a signal meeting a threshold value. In still other embodiments, analysis involves a comparison of more than one signal (e.g., examining differences in signal level, calculating ratios, etc.). In preferred embodiments, a result (i.e., a determination of genotype at that locus, such as homozygous Allele 1 or Allele 2, heterozygous, Indeterminate) is determined when the processed data yields or corresponds to a value that has been predetermined to be indicative of a particular SNP result.

In some embodiments, the SNP results data from one plate are compared with the SNP results data from another plate. In other embodiments, SNP results data generated by one SNP analysis source method are compared to SNP results data generated by another SNP analysis method (e.g., INVADER assay results are compared to gene chip data).

In some embodiments, analysis results are displayed. In other embodiments, the analysis results are exported (e.g., sent to a printer or a file, or to a further process step) without display. In preferred embodiments, results are displayed on a screen. In particularly preferred embodiments, results are displayed in a plate viewer. In some preferred embodiments, the plate viewer is displayed in a new window. In particularly preferred embodiments, the window is printable.

In some embodiments, the user may select a particular target result from the display of results and view the information in fields. In some embodiments, selection of an entry creates a display of the fields for that entry. In some embodiments, all the fields of the record in an association database are shown. In other embodiments, a subset of the fields is shown. In preferred embodiments, fields in results records include but are not limited to results of the analysis (e.g., presence of a particular nucleic acid sequence), the entered or imported raw input assay data (e.g., measured fluorescence, measured peaks, etc.), or the analyzed input assay data by which the allele determination was made (e.g., calculated differences in signal level, calculated ratios). In preferred embodiments, a field for user comments is included. In particularly preferred embodiments, the user comment field is editable after a SNP result has been obtained. In further particularly preferred embodiments, changes in a SNP result record may be saved by the user to that record or to a version of that record after a comment field is edited.

In some embodiments, the user selects which field of the result record assigned to that cell will be displayed in the cell. In some embodiments, different fields from each result record may be displayed in each of the different cells. In other embodiments, the cells are coordinated so that the same field from each SNP result record is displayed in each assigned cell. In a preferred embodiment, the user can globally change the fields displayed in all wells (e.g., through the use of a menu), such that all of the cells can be changed at one time to display the same field from each different result record.

In preferred embodiments, the fields are displayed in a new window. In other embodiments, the fields are exported (e.g., sent to a printer or a file, or to a further process step) without display. In a preferred embodiment, the fields are displayed in a printable window. In some embodiments, one or more fields will contain one ore more local or Internet links (e.g., hypertext links or URLs). In preferred embodiments, the user can click on links to bring up the corresponding content.

In some embodiments, there is a code to visually distinguish test results and control reaction results (e.g., ‘no target’ controls or other controls). In preferred embodiments, the code is a color code.

In some embodiments, the fields are exportable to a spreadsheet file or worksheet (e.g., in Microsoft Excel). In some embodiments, result data are exported to a worksheet by field content (e.g., one worksheet with all allele calls, one worksheet with all calculated ratios of signals, one worksheet with all raw input fluorescence measurements). In other embodiments, results data are exported, all data is exported to a single worksheet, with data grouped according to the well with which it corresponds. In preferred embodiments, the user has the option (e.g., through a menu or window) of selecting a variety ways in which the results data are sorted and/or grouped for export to a spreadsheet.

In preferred embodiments, following verification, assays for the detection of a given target are tested on a plurality of additional individuals. Data from additional assays is combined with information obtained from database searches. In preferred embodiments, the result is a revised reliability score for the target. In particularly preferred embodiments, data from additional analysis (e.g., results generated by an investigator using the methods and systems of the present invention) is used to update or amend an association database containing information about the given target.

III. Detection Assay Formats

The present invention contemplates a variety of formats for nucleic acid detection assays. Exemplary, non-limiting formats are described below.

A. Lateral Flow Strip Assay

In some preferred embodiments of the present invention, nucleic acid detection is performed via a lateral flow strip assay. The lateral flow strip assay format is suitable for a variety of nucleic acid detection formats, including, but not limited to, those described herein. The lateral flow strip assay is described herein in the context of the INVADER assay. However, one skilled in the art knows well how to adapt additional assays to the lateral flow strip format.

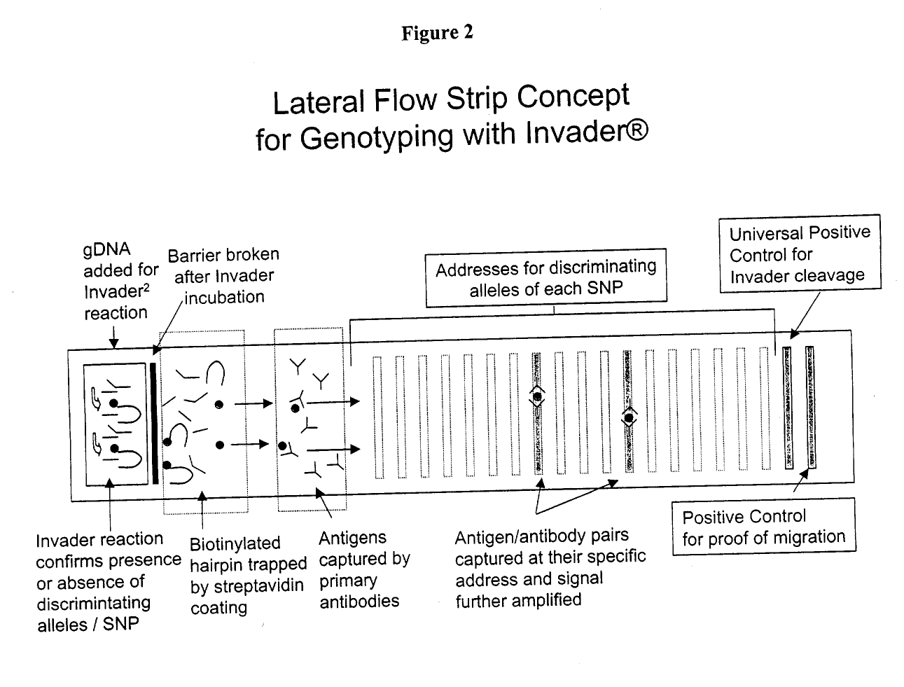

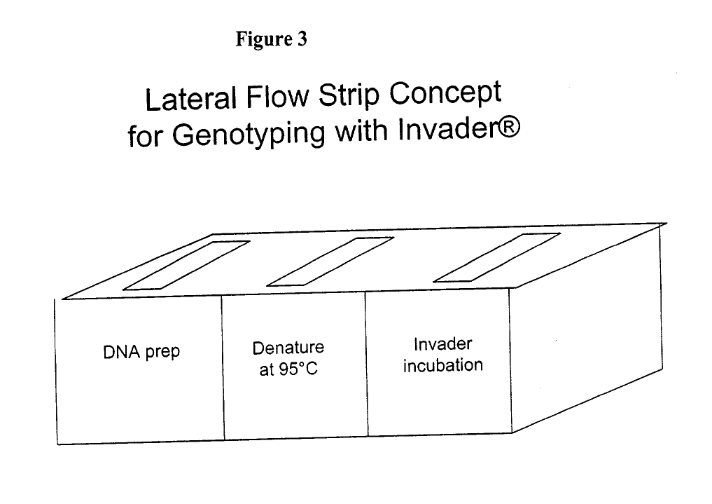

An overview of the lateral flow strip is shown in FIG. 2. Briefly, a first well contains all of the components necessary for the nucleic acid detection assay (e.g., INVADER assay). The assay is performed in this well. FIG. 3 provides a schematic of the incubation chamber utilized in sample preparation and assay performance. For example, in preferred embodiments of the present invention, a representative wheat sample is added to the reaction well of the strip. In some embodiments, the sample is homogenized. In some embodiments (e.g., those involving the determination of wheat genotype), nucleic acid is extracted from the wheat samples prior to analysis. In other embodiments, (e.g., those where it is desirable to determine the presence of contaminants), samples are utilized without extraction.

In preferred embodiments, one component of the detection assay is labeled. It is preferred that the labeled component is altered in the detection assay in such a way as to allow discrimination of the labeled component in the presence or absence of target nucleic acid. For example, in some embodiments, an INVADER assay detection cassette is labeled with a detectable label (e.g., antigen), as well as a moiety suitable for sorting and immobilization (e.g., biotin). Cleavage of the cassette releases the label.

Upon the completion of the detection assay, the barrier in between the reaction well and the remainder of the lateral flow strip is broken and the sample flows into a nucleic acid capture well. The labeled nucleic acid (e.g., the cleaved INVADER detection cassette) containing a first binding partner (e.g., biotin) sticks to the well, which is coated with a moiety specific for the first binding pair (e.g., strepavidin). All of the detection cassettes stick to the capture well. Only cassettes that have been cleaved due to the presence of the target nucleic acid release their labels into the remainder of the strip.

In some embodiments, released labels (e.g., antigens) flow into a label capture well. In the capture well, antigens are captured by specific antibodies. In other embodiments, labels flow directly into the detection section of the strip.

The labels (e.g., bound by their specific antibodies) next flow into the detection section of the strip. The detection section contains a series of addresses specific for a given nucleic acid. In some embodiments, addresses are specified via an antibody specific for the antigen. In other embodiments, addresses are specified via an antibody specific for a first antibody specifically bound to a given antigen. Antigens are allowed to migrate to their specific addresses for a suitable period of time. Followed addressing, antigens are detected using any suitable method (e.g., including, but not limited to, the use of a labeled secondary antibody specific for all of the antigen specific antibodies). In some preferred embodiments, the strip additionally includes a universal positive control for the detection assay, as well as a positive control for proof of migration. The universal positive control is read using the methods used to detect a reaction. The migration control may be any detectable reagent (e.g., a visible dye) that is read via the presence of a specific color.

Adresses are “read” via any suitable method. For example, in some embodiments, a user visually inspects the strip. In other embodiments, the strip is read in an automated manner (e.g., via computer and computer softwar). In some embodiments, the software is linked to the Internet, allowing for the distribution of information to a variety of interested parties (e.g., the farmer, the CGC, the Canadian Wheat Board, and customers).

B. Other Formats

The present invention is not limited to the the lateral flow strip format. Any suitable format may be utilized. For example, many of the hybridization based detection assays described herein (e.g., INVADER assay) are suitable for use in solid support formats such as arrays or dry-down plates (e.g., microtiter plates).

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

We claim:

1. A method of determining the grade of a wheat sample, comprising:
a) providing
i) a wheat sample;
ii) detection assay components suitable for the detection of three or more properties of said wheat sample; and
b) performing a detection assay with said detection assay components and said wheat sample.

2. The method of claim 1, wherein said detection assay components are suitable for the detection of 5 or more properties of said wheat sample.

3. The method of claim 1, wherein said detection assay components are suitable for the detection of 10 or more properties of said wheat sample.

4. The method of claim 1, wherein said detection assay components are suitable for the detection of 15 or more properties of said wheat sample.

5. The method of claim 1, further comprising the step of determining the grade of said wheat sample based on the results of said detection assay.

6. The method of claim 1, wherein said three or more properties are selected from the group consisting of presence of contaminating organisms, presence of contaminating wheat, presence of contaminating plants, presence of contaminating seeds, and presence of genetically modified organisms.

7. The method of claim 6, wherein said contaminating wheat is a different variety of wheat than said wheat sample.

8. The method of claim 6, wherein said contaminating organisms are selected from the group consisting of micro organisms and macro organisms.

9. The method of claim 8, wherein said micro organisms are selected from the group consisting of ergot, sclerotinia, fusarium, smut, mildew, streak mold, and smudge.

10. The method of claim 8, wherein said macro organisms are selected from the group consisting of grasshopper, sawfly, midge, and army worm.

11. The method of claim 6, wherein said contaminating plants are selected from the group consisting of grass, rye, barley, tritcale, oats, oat groats, and wild oat groats.

12. The method of claim 6, wherein said contaminating seeds are selected from the group consisting of ragweed, tartary buckwheat, rye grass, and wild oats.

13. The method of claim 1, wherein said detection assay is selected from the group consisting of a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, and a sandwich hybridization assay.

14. The method of claim 1, wherein said assay is performed using a lateral flow strip.

15. The method of claim 13, wherein said hybridization assay is an INVADER assay.

16. A method of detecting contaminating wheat in a wheat sample, comprising:
a) providing
i) a wheat sample suspected of containing contaminating wheat;
ii) detection assay components suitable for the detection of one or more types of contaminating wheat is said sample, wherein said contaminating wheat is a different variety of wheat than said wheat sample; and
b) performing a detection assay with said detection assay components and said wheat sample.

17. The method of claim 16, wherein said detection assay components are suitable for the detection of 3 or more types of contaminating wheat is said sample.

18. The method of claim 16, wherein said detection assay components are suitable for the detection of 5 or more types of contaminating wheat is said sample.

19. The method of claim 16, further comprising the step of determining the number and identity of said contaminating wheat present in said wheat sample.

20. The method of claim 19, further comprising the step of determining the grade of said wheat sample based on the results of said detection assay.

21. The method of claim 1, further comprising the step of detecting three or more properties of said wheat sample, wherein said three or more properties are selected from the group consisting of presence of contaminating organisms, presence of contaminating plants, presence of contaminating seeds, and presence of genetically modified organisms.

22. The method of claim 21, wherein said contaminating organisms are selected from the group consisting of micro organisms and macro organisms.

23. The method of claim 22, wherein said micro organisms are selected from the group consisting of ergot, sclerotinia, fusarium, smut, mildew, streak mold, and smudge.

24. The method of claim 22, wherein said macro organisms are selected from the group consisting of grasshopper, sawfly, midge, and army worm.

25. The method of claim 21, wherein said contaminating plants are selected from the group consisting of grass, rye, barley, tritcale, oats, oat groats, and wild oat groats.

26. The method of claim 21, wherein said contaminating seeds are selected from the group consisting of ragweed, tartary buckwheat, rye grass, and wild oats.

27. The method of claim 16, wherein said detection assay is selected from the group consisiting of a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, and a sandwich hybridization assay.

28. The method of claim 16, wherein said assay is performed using a lateral flow strip.

29. A system for the grading of wheat, comprising:
a) a detection assay component configured for the generation of nucleic acid information for three or more properties of a wheat sample; and
b) an information distribution component configured for the distribution of said nucleic acid information.

30. The system of claim 29, wherein said three or more properties are selected from the group consisting of presence of contaminating organisms, presence of contaminating wheat, presence of contaminating plants, presence of contaminating seeds, and presence of genetically modified organisms.

31. The system of claim 30, wherein said contaminating wheat is a different variety of wheat than said wheat sample.

32. The system of claim 30, wherein said contaminating organisms are selected from the group consisting of micro organisms and macro organisms.

33. The system of claim 32, wherein said micro organisms are selected from the group consisting of ergot, sclerotinia, fusarium, sumt, mildew, streak mold, and smudge.

34. The system of claim 32, wherein said macro organisms are selected from the group consisting of grasshopper, sawfly, midge, and army worm.

35. The system of claim 30, wherein said contaminating plants are selected from the group consisting of grass, rye, barley, tritcale, oats, oat groats, and wild oat groats.

36. The system of claim 30, wherein said contaminating seeds are selected from the group consisting of ragweed, tartary buckwheat, rye grass, and wild oats.

37. The system of claim 29, wherein said detection assay component comprises reagent for performing a detection assay selected from the group consisting of a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, and a sandwich hybridization assay.

38. The system of claim 29, wherein said detection assay component is a lateral flow strip.

39. The system of claim 29, wherein said an information distribution component comprises a computer system, said computer system comprising a computer processor and computer memory.

40. The system of claim 39, wherein said computer processor and computer memory are in communication with the Internet.

41. The system of claim 39, wherein said computer system is in possession of a farmer.

42. The system of claim 39, wherein said computer system is in possession of a distributor.

43. The system of claim 39, wherein said computer system is in possession of a customer.


SEQUENCE LISTING (4 sequences)

SEQ ID NO: 1 - DNA, 10 bp, Artificial Sequence
  Synthetic
cgcgccgagg

SEQ ID NO: 2 - DNA, 14 bp, Artificial Sequence
  Synthetic
atgacgtggcagac

SEQ ID NO: 3 - DNA, 12 bp, Artificial Sequence
  Synthetic
acggacgcggag

SEQ ID NO: 4 - DNA, 11 bp, Artificial Sequence
  Synthetic
tccgcgcgtcc